(19) United States
(12) Patent Application Publication
(10) Pub. No.: US 2025/0011792 A1
(43) Pub. Date: Jan. 9, 2025

(54) IRAP INHIBITORS FOR USE IN THE TREATMENT OF INFLAMMATORY DISEASES

(71) Applicants: INSTITUT NATIONAL DE LA SANTÉ ET DE LA RECHERCHE MÉDICALE, Paris (FR); ASSISTANCE PUBLIQUE-HÔPITAUX DE PARIS (APHP), Paris (FR); FONDATION IMAGINE, Paris (FR); UNIVERSITÉ PARIS CITÉ, Paris (FR); CENTRE NATIONAL DE LA RECHERCHE SCIENTIFIQUE, Paris (FR)

(72) Inventors: Olivier HERMINE, Paris (FR); Mirjana WEIMERSHAUS, Paris (FR); Thiago TROVATI MACIEL, Paris (FR); Peter VAN ENDERT, Paris (FR); Michael DUSSIOT, Paris (FR); Rachel RIGNAULT-BRICARD, Paris (FR); Caroline CARVALHO, Paris (FR)

(21) Appl. No.: 18/709,695

(22) PCT Filed: Nov. 30, 2022
(86) PCT No.: PCT/EP2022/083902

§ 371 Date: May 13, 2024

(30) Foreign Application Priority Data
Dec. 1, 2021 (EP) 21306677.2

(51) Int. Cl. C12N 15/113; A61K 31/351; A61K 31/381; A61K 31/4433; A61K 31/4709; A61K 38/07; A61K 38/12

(52) U.S. Cl. CPC C12N 15/1137; A61K 31/351; A61K 31/381; A61K 31/4433; A61K 31/4709; A61K 38/07; A61K 38/12; C12N 2310/11; C12N 2310/14; C12Y 304/11003

(57) ABSTRACT

Upon activation, mast cells rapidly release preformed inflammatory mediators from large cytoplasmic granules via regulated exocytosis. This acute degranulation is followed by a late activation phase involving synthesis and secretion of cytokines, growth factors and other inflammatory molecules via the constitutive pathway that remains ill-defined. Here the inventors describe a role for an insulin-responsive vesicle-like endosomal compartment, marked by insulin-regulated aminopeptidase (IRAP), in the secretion of TNF-α and IL-6 in mast cells and macrophages. IRAP-deficient mice are protected from TNF-dependent kidney injury and inflammatory arthritis. In the absence of IRAP, TNF fails to be efficiently exported from the Golgi. Chemical targeting of IRAP+ endosomes reduced pro-inflammatory cytokine secretion thereby highlighting this compartment as a promising target for the therapeutic control of inflammation. Thus the present invention relates to the use of IRAP inhibitors for the treatment of inflammatory diseases

### FIELD OF THE INVENTION

The present invention is in the field of medicine, in particular inflammatory diseases.

### BACKGROUND OF THE INVENTION

Mast cells are phylogenetically ancient innate immune cells residing in most connective and mucosal tissues. Their prominent characteristic are large cytoplasmic granules filled with proteases, histamine, serotonin, cytokines and inflammatory mediators that are rapidly released upon signaling through specific cell surface receptors, including FcεR, complement receptors, TLRs, and GPCRs. Moreover, in a second phase after activation, mast cells synthesize and secrete large amounts of pro- and anti-inflammatory cytokines, chemokines and growth factors, prostaglandins and leukotrienes. Interestingly, certain stimuli, such as LPS and IL-10 exclusively activate secretion of de novo synthesized mediators without triggering release of preformed granules¹⁻³. While our understanding of the processes involved in the biogenesis and triggered release of preformed granule content in anaphylactic reactions has been advanced in recent years, much remains to be learned about the immunomodulatory roles of mast cell-derived cytokines, chemokines and growth factors, including the regulation of their secretion^{3,4}.

Mast cell-derived cytokines, chemokines and growth factors can act in autocrine, paracrine, local and systemic fashion, and are involved in physiological and protective processes such as angiogenesis, wound healing and the immune defense against bacteria and viruses, as well as in pathological processes such as autoimmune, metabolic and neurological disorders, fibrosis and cancer⁵. With regards to the role of mast cells in disease-related settings, pro-inflammatory cytokines play a prime role. Especially, mast cell-derived TNF-α and IL-6 and have been in the focus of numerous studies. They have been reported to chemotactically attract neutrophils and macrophages, to upregulate adhesion molecules in endothelial cells⁶⁻⁸, modulate DC functions^{9,10}, promote colitis¹¹, mediate cisplatin-induced kidney injury¹², participate in inflammatory arthritis¹³, and control airway hyperreactivity, inflammation, and Th2 recruitment and cytokine production in a murine antigen-induced asthma model¹⁴.

Targeting cytokine synthesis and secretion in the late or chronic activation phase in mast cells might therefore represent a promising therapeutic strategy to prevent adverse mast cell-related inflammatory reactions and actively shape the immuno-modulatory responses of these versatile cells.

Of note, several hundreds of biological compounds with various functions have been identified in the mast cell secretome^{15,16} Some of these have opposed physiological functions, suggesting that secretory pathways and products in mast cells may be temporally and spatially regulated. Exocytic mechanisms, i.e. active vesicular transport resulting in compound release, are present in all eukaryotic cells. While products and biological functions of exocytosis vary largely between cell types, the underlying pathways and trafficking machinery are highly conserved. Two major pathways can be mechanistically distinguished, referred to as regulated and constitutive secretion, respectively. A few recent studies have started to elucidate the mechanistic aspects that segregate constitutive cytokine secretion from regulated granule exocytosis^{17,20}. This discrimination has been advanced by the identification of the distinct Soluble N-ethylmaleimide-sensitive factor attachment protein receptors (SNARE) proteins involved in either pathway. SNARE proteins confer membrane identity and control fusion of lipid bilayers of distinct compartments, as each v (vesicular)-SNARE family member can form complexes only with a limited set of t (target)-SNARE partners.

In the constitutive pathway in macrophages, where cytokine secretion has been studied best, following translation, cytokines traffic from the ER through the Golgi stacks, from where they are exported in syntaxin (Stx) 6 SNARE-identified vesicles²¹. These post-Golgi carriers then fuse with Rab11+ VAMP3+ recycling endosomes and are further transported to the plasma membrane (PM)^{21,22}. The major SNARE complex required for VAMP3 vesicle fusion with the PM is composed of SNAP23 and Stx4 in murine mast cells and macrophages^{19,23}. As specific sorting signals routing cargo from the trans-Golgi network (TGN) into constitutive secretion are yet to be identified, this pathway is considered as a “default” pathway²⁴.

In contrast, proteins destined to packing into secretory lysosome-related granules are actively sorted away in the Golgi to form immature secretory granules. These pre-granules undergo a series of fusion and fission events which result in removal of mis-sorted cargo and condensation giving rise to mature granules stored in the cytoplasm²⁵.

Release of secretory granules in mast cells requires activation via inflammatory cell surface receptors, including FcεR, FcγR, TLRs, complement receptors and other GPCRs, and is mediated through elevation of intracellular Ca²⁺ levels and activation of PKC^{26,27}. The main SNARE protein mediating attachment and fusion of the secretory granules with the plasma membrane is VAMP8. Of note, the same SNARE complex as in the constitutive pathway, composed of SNAP23 and Stx4, is employed for secretory granule attachment and fusion at the plasma membrane^{19,28,29}.

Interestingly, limited amounts of preformed cytokines including TNF-α are found in secretory granules⁶ and, at least in human mast cell lines, have been suggested to traffic there by re-endocytosis from the extracellular space rather than after direct TGN-sorting to these granules³⁰.

Moreover, a distinct compartment of recycling vesicles has been described in mast cells³¹. These vesicles are identified by the expression of insulin-regulated aminopeptidase (IRAP) and, in resting cells, exhibit a prevailing cytosolic distribution near the ER-Golgi intermediate compartment (ERGIC) from where they undergo slow recycling to the PM. Upon signal transduction through the FcεR, IRAP rapidly translocates to the plasma membrane, where it may participate in signal transduction events. Importantly, IRAP endosome mobilization is mechanistically segregated from the exocytosis of secretory granules³¹. This suggests that IRAP and granule-contained mediators such as histamine are present in distinct compartments, but the precise relationship of IRAP to the different secretory pathways in mast cell is unknown. The possible positive or negative regulation of the degranulation process by IRAP endosomes, for instance, has not been elucidated by Liao et al. due to the absence of IRAP-invalidated cells or animals in the study. Thus, considering the intersection of exocytosis with endocytic and recycling pathways, as well as the importance of their coordination for mast cell physiology, we set out to investigate the communication of IRAP-containing endosomes with the aforementioned exocytic pathways.

IRAP endosomes have mainly been described as Glucose-transporter (Glut) 4 storage vesicles (GSV) in adipocytes and muscle cells where they have been extensively studied with respect to their function in insulin-stimulated Glut4 trafficking^{32,33} In insulin-responsive cells, upon an activating signal through the insulin receptor, the dynamic retention of GSV in the cytosol is released, which allows them to traffic to the cell surface, thereby inserting Glut4, IRAP and other transmembrane GSV proteins into the plasma membrane^{34,35}. From here, IRAP and Glut4 are re-internalized into sorting endosomes, where they have been shown to interact with the retromer complex that promotes their deviation from the degradative late endosomal/lysosomal pathway and retrieves them for retrograde TGN³⁶, the GSV assembly and budding site.

Of note, in contrast to the relatively restricted Glut4 expression patterns to insulin-responsive tissues, IRAP-containing endosomes are widely expressed amongst cell types and tissues, where they are mobilized by cell-specific surface receptor signaling and employed for various cell type-specific functions³⁷⁻³⁹. In this line, with regards to immune cells, the trafficking of IRAP endosomes has recently been recognized to intersect with and regulate phagosome maturation and MHC—I cross-presentation in dendritic cells⁴⁰⁻⁴³, activation of TLR9⁴⁴, as well as endo- and exocytic trafficking in T cells for the supply of TCR signaling components and optimal TCR signaling⁴⁵.

### SUMMARY OF THE INVENTION

The present invention is defined by the claims. In particular, the present invention relates to the use of IRAP inhibitors for the treatment of inflammatory diseases.

### DETAILED DESCRIPTION OF THE INVENTION

Upon activation, mast cells rapidly release preformed inflammatory mediators from large cytoplasmic granules via regulated exocytosis. This acute degranulation is followed by a late activation phase involving synthesis and secretion of cytokines, growth factors and other inflammatory molecules via the constitutive pathway that remains ill-defined. Here the inventors describe a role for an insulin-responsive vesicle-like endosomal compartment, marked by insulin-regulated aminopeptidase (TRAP), in the secretion of TNF-α and IL-6 in mast cells and macrophages. IRAP-deficient mice are protected from TNF-dependent kidney injury and inflammatory arthritis. In the absence of IRAP, TNF fails to be efficiently exported from the Golgi. Subsequently, reduced co-localization of VAMP3+ endosomes with Stx4 was observed, while VAMP8-dependent exocytosis of secretory granules was facilitated. Chemical targeting of IRAP+ endosomes reduced pro-inflammatory cytokine secretion thereby highlighting this compartment as a promising target for the therapeutic control of inflammation.

Accordingly, the first object of the present invention relates to a method of treating an inflammatory disease in a patient in need thereof comprising administering to the patient a therapeutically effective amount of an IRAP inhibitor.

As used herein, the term “inflammatory disease” has its general meaning in the art and refers to a disease that is associated with inflammation mediated by at least one proinflammatory cytokine.

As used herein, the term “pro-inflammatory cytokine” has its general meaning in the art and refers to a cytokine that promote inflammation. Pro-inflammatory cytokines, include, for example, IL-6, IL-8, TNF-alpha, IL1-alpha, IL1-beta, IFN-alpha, IFN-beta, IFN-gamma, IL-10, IL12, IL-23, IL17, and IL18. IL-1, IL-2, IL-3, IL-4, IL-5, IL-7, IL-8, IL-9, IL-11, IL-12, TNFα, TNFβ, interferon 7, GCSF, GMCSF or MCSF.

As used herein, the term “treatment” or “treat” refers to both prophylactic or preventive treatment as well as curative or disease modifying treatment, including treatment of patients at risk of contracting the disease or suspected to have contracted the disease as well as patients who are ill or have been diagnosed as suffering from a disease or medical condition, and includes suppression of clinical relapse. The treatment may be administered to a subject having a medical disorder or who ultimately may acquire the disorder, in order to prevent, cure, delay the onset of, reduce the severity of, or ameliorate one or more symptoms of a disorder or recurring disorder, or in order to prolong the survival of a subject beyond that expected in the absence of such treatment. By “therapeutic regimen” is meant the pattern of treatment of an illness, e.g., the pattern of dosing used during therapy. A therapeutic regimen may include an induction regimen and a maintenance regimen. The phrase “induction regimen” or “induction period” refers to a therapeutic regimen (or the portion of a therapeutic regimen) that is used for the initial treatment of a disease. The general goal of an induction regimen is to provide a high level of drug to a patient during the initial period of a treatment regimen. An induction regimen may employ (in part or in whole) a “loading regimen”, which may include administering a greater dose of the drug than a physician would employ during a maintenance regimen, administering a drug more frequently than a physician would administer the drug during a maintenance regimen, or both. The phrase “maintenance regimen” or “maintenance period” refers to a therapeutic regimen (or the portion of a therapeutic regimen) that is used for the maintenance of a patient during treatment of an illness, e.g., to keep the patient in remission for long periods of time (months or years). A maintenance regimen may employ continuous therapy (e.g., administering a drug at a regular interval, e.g., weekly, monthly, yearly, etc.) or intermittent therapy (e.g., interrupted treatment, intermittent treatment, treatment at relapse, or treatment upon achievement of a particular predetermined criteria [e.g., disease manifestation, etc.]).

In particular, the IRAP inhibitor of the present invention is particularly suitable for reducing secretion of pro-inflammatory cytokine, in particular by mast cells.

In some embodiments, the inflammatory disease is selected from the group consisting of arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma, systemic scleroderma, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (MPGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, including vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus, optionally Pemphigus vulgaris, Pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis and oophoritis, primary hypothyroidism, hypoparathyroidism, autoimmune thyroiditis, Hashimoto's disease, chronic thyroiditis (Hashimoto's thyroiditis); subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal garnmopathy of undetermined significance, MGUS, peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, infertility due to antispermatozoan antobodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

In some embodiments, the patient suffers from an allergic disorder. As used herein, “allergic disorder” refers to any disorder resulting from antigen activation of mast cells that results in an “allergic reaction” or state of hypersensitivity and influx of inflammatory and immune cells. Those disorders include without limitation: systemic allergic reactions, systemic anaphylaxis or hypersensitivity responses, anaphylactic shock, drug allergies, and insect sting allergies; respiratory allergic diseases, such asthma, hypersensitivity lung diseases, hypersensitivity pneumonitis and interstitial lung diseases (ILD), ILD associated with rheumatoid arthritis, or other autoimmune conditions); rhinitis, hay fever, conjunctivitis, and allergic rhinoconjunctivitis.

In some embodiments, the patient suffers from asthma. As used herein, the term “asthma” refers to an inflammatory disease of the respiratory airways that is characterized by airway obstruction, wheezing, and shortness of breath.

In some embodiments, the patient suffers from anaphylaxis. As used herein, the term “anaphylaxis” refers to a life threatening allergic reaction characterized by decreased blood pressure, respiratory failure with bronchoconstriction, and skin rash due to release of mediators from cells such as mast cells.

In some embodiments, the inflammatory diseases is secondary to therapeutic treatment, in particular a treatment with an immune checkpoint inhibitor. As used herein, the term “immune checkpoint inhibitor” has its general meaning in the art and refers to any compound inhibiting the function of an immune inhibitory checkpoint protein. Inhibition includes reduction of function and full blockade. Preferred immune checkpoint inhibitors are antibodies that specifically recognize immune checkpoint proteins. In some embodiments, the immune checkpoint inhibitor is an antibody selected from the group consisting of anti-CTLA4 antibodies, anti-PD-1 antibodies, anti-PD-L1 antibodies, anti-PD-L2 antibodies anti-TIM-3 antibodies, anti-LAG3 antibodies, anti-B7H3 antibodies, anti-B7H4 antibodies, anti-BTLA antibodies, and anti-B7H6 antibodies.

In some embodiments, the patient suffers from chemotherapy induced inflammation. Chemotherapy is a category of cancer treatment that uses chemical substances, especially one or more anti-cancer drugs (chemotherapeutic agents) that are given as part of a standardized chemotherapy regimen. Chemotherapy may be given with a curative intent, or it may aim to prolong life or to reduce symptoms. Chemotherapies include alkylating agent chemotherapy, anti-metabolite chemotherapy, anti-microtubule chemotherapy, topoisomerase inhibitor chemotherapy, and cytotoxic antibiotic chemotherapy. In certain aspects the chemotherapy is an alkylating chemotherapy. Alkylating chemotherapy includes, but is not limited to nitrogen mustards, nitrosoureas, tetrazines, aziridines, and cisplatins. In particular, the method of the present invention is particularly suitable for the treatment of cisplatin-induced kidney inflammation.

As used herein, the term “IRAP” has its general meaning in the art and refers to insulin-regulated membrane aminopeptidase. The term is also known as leucyl-cystinyl aminopeptidase, insulin-responsive aminopeptidase. An exemplary amino acid sequence is represented by SEQ ID NO:1.

>sp|Q9UIQ6|LCAP_HUMAN Leucyl-cystinyl amino-
peptidase OS = Homo sapiens OX = 9606 GN = LNPEP
PE = 1 SV = 3
SEQ ID NO: 1
MEPFTNDRLQLPRNMIENSMFEEEPDVVDLAKEPCLHPLEPDEVEYEPRG
SRLLVRGLGEHEMEEDEEDYESSAKLLGMSFMNRSSGLRNSATGYRQSPD
GACSVPSARTMVVCAFVIVVAVSVIMVIYLLPRCTFTKEGCHKKNQSIGL
IQPFATNGKLFPWAQIRLPTAVVPLRYELSLHPNLTSMTFRGSVTISVQA
LQVTWNIILHSTGHNISRVTFMSAVSSQEKQAEILEYAYHGQIAIVAPEA
LLAGHNYTLKIEYSANISSSYYGFYGFSYTDESNEKKYFAATQFEPLAAR
SAFPCFDEPAFKATFIIKIIRDEQYTALSNMPKKSSVVLDDGLVQDEFSE
SVKMSTYLVAFIVGEMKNLSQDVNGTLVSIYAVPEKIGQVHYALETTVKL
LEFFQNYFEIQYPLKKLDLVAIPDFEAGAMENWGLLTFREETLLYDSNTS
SMADRKLVTKIIAHELAHQWFGNLVTMKWWNDLWLNEGFATFMEYFSLEK
IFKELSSYEDFLDARFKTMKKDSLNSSHPISSSVQSSEQIEEMFDSLSYF
KGSSLLLMLKTYLSEDVFQHAVVLYLHNHSYASIQSDDLWDSFNEVINQT
LDVKRMMKTWTLQKGFPLVTVQKKGKELFIQQERFFLNMKPEIQPSDTSY
LWHIPLSYVTEGRNYSKYQSVSLLDKKSGVINLTEEVLWVKVNINMNGYY
IVHYADDDWEALIHQLKINPYVLSDKDRANLINNIFELAGLGKVPLKRAF
DLINYLGNENHTAPITEALFQTDLIYNLLEKLGYMDLASRLVTRVFKLLQ
NQIQQQTWTDEGTPSMRELRSALLEFACTHNLGNCSTTAMKLFDDWMASN
GTQSLPTDVMTTVFKVGAKTDKGWSFLLGKYISIGSEAEKNKILEALASS
EDVRKLYWLMKSSLNGDNFRTQKLSFIIRTVGRHFPGHLLAWDFVKENWN
KLVQKFPLGSYTIQNIVAGSTYLFSTKTHLSEVQAFFENQSEATFRLRCV
QEALEVIQLNIQWMEKNLKSLTWWL

As used herein, an “IRAP inhibitor” has its general meaning in the art and refers to any compound that inhibits the activity or expression of IRAP. The compound may be a competitive, non-competitive, orthosteric, allosteric, or partial inhibitor. In some embodiments, the inhibitor is a molecule that inhibits the enzyme activity of IRAP for example by binding the active site, or competing with the enzyme substrate or co-effector or signalling mechanism. The inhibitor may be specific for IRAP and only have some low level inhibitory activity against other receptors (for example, a Ki of greater than about 50 μM or 100 μM, preferably 1 mM against other receptors as measured using an assay as described herein, or for example a Ki against other receptors at least 10× greater than the Ki against IRAP). The enzymatic activities of IRAP may be determined by the hydrolysis of the synthetic substrate Leu-MCA (Sigma-Aldrich, Missouri, USA) monitored by the release of a fluorogenic product, MCA, at excitation and emission wavelengths of 380 and 440 nm, respectively according to Albiston et al. 2008 The FASEB Journal 22:4209-4217 or other method described herein. Inhibitors of IRAP are known in the art. For example, IRAP inhibitors described in Albiston et al. (2008) The FASEB Journal 22:4209-4217; Albiston et al. (2011), British Journal of Pharmacology, 164:37-47, Albiston, et al. J Biol. Chem. 276, 48263-48266; U.S. Pat. No. 6,066,672; Albiston, et al. Pharmacol. Ther. 116, 417-427; Axen, et al. (2006) J. Pept. Sci. 12, 705-713; Albiston et al. (2010) Molecular Pharmacology, 78(4): 600-607; Mountford, et al. (2014) J Med Chem 57(4): 1368-1377; Andersson et al. J Med Chem (2010) 53, 8059, Andersson et al. (2011) J Med Chem 54(11):3779-3792; WO2009065169; WO2010001079; WO 2000012544; US 2004/0086510; WO 2003/01 1304; and WO2006026832, and may be useful in the present invention.

In some embodiments, the IRAP inhibitor of the present invention has a structure according to Formula (I):

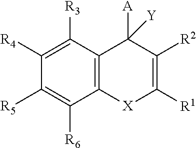

A is aryl, heteroaryl carbocyclyl or heterocyclyl, each of which may be optionally substituted, when R1 is NHCOR₈; or quinolinyl, isoquinolinyl, cinnolinyl, quinazolinyl, quinoxalinyl, 1,8-naphthyridyl, phthalazinyl or pteridinyl, each of which may be optionally substituted, when R1 is NR₇R₈, NHCOR₈, N(COR₈)₂, N(COR₇)(COR₈), N═CHOR₈ or N═CHR₈;
X is 0, NR′ or S, wherein R′ is hydrogen, optionally substituted alkyl, optionally substituted alkenyl, optionally substituted alkynyl, optionally substituted aryl, optionally substituted acyl, optionally substituted heteroaryl, optionally substituted carbocyclyl or optionally substituted heterocyclyl;
R7 and R8 are independently selected from hydrogen, optionally substituted alkyl, optionally substituted aryl, or R7 and R8, together with the nitrogen atom to which they are attached form a 3-8-membered ring which may be optionally substituted;
R2 is CN, C02R9, C(0)0(0)R9, C(0)R9 or C(O)NR9R10 wherein R9 and R10 are independently selected from alkyl, alkenyl, alkynyl, aryl, heteroaryl, carbocyclyl, heterocyclyl, each of which may be optionally substituted, and hydrogen; or R9 and R10 together with the nitrogen atom to which they are attached, form a 3-8-membered ring which may be optionally substituted;
R3-R6 are independently selected from hydrogen, halo, nitro, cyano alkyl, alkenyl, alkynyl, aryl, heteroaryl, heterocyclyl, carbocyclyl, hydroxy, alkoxy, alkenyloxy, alkynyloxy, alkynyloxy, aryloxy, heteroaryloxy, heterocyclyloxy, amino, acyl, acyloxy, carboxy, carboxyester, methylenedioxy, amido, thio, alkylthio, alkenylthio, alkynylthio, arylthio, heteroarylthio, heterocyclylthio, carbocyclylthio, acylthio and azido, each of which may be optionally substituted where appropriate, or any two adjacent R3-R6, together with the atoms to which they are attached, form a 3-8-membered ring which may be optionally substituted; and
Y is hydrogen or C1-10 alkyl, or a pharmaceutically acceptable salt or solvate thereof.

In some embodiments, A is optionally substituted heteroaryl when R¹ is NHCOR8. In some embodiments, A is pyridinyl.

In some embodiments, X is 0.

In some embodiments, R2 is CO₂R9.

In some embodiments, R5 is hydroxyl.

In some embodiments, the IRAP inhibitor has the structure:

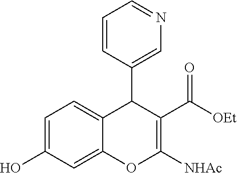 [SMILES: CCOC(=O)C1=C(NC(C)=O)Oc2cc(O)ccc2C1c1cccnc1]

In some embodiments, the IRAP inhibitor of the present invention has a structure according to Formula (II):

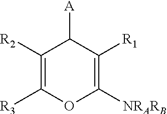

wherein:
A is selected from alkyl, alkenyl, alkynyl, aryl, arylalkyl, heteroaryl, heteroarylalkyl, carbocyclyl, carbocyclylalkyl, each of which may be optionally substituted;
RA and RB are independently selected from hydrogen, alkyl and acyl;
R1 is selected from CN or CO2RC;
R2 is selected from CO2RC and acyl;
R3 is selected from alkyl, alkenyl, alkynyl, aryl, arylalkyl, heteroaryl, heteroarylalkyl, carbocyclyl, carbocyclylalkyl, each of which may be optionally substituted; or
R2 and R3 together form a 5-6-membered saturated keto-carbocyclic ring:

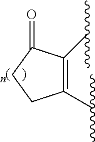

wherein n is 1 or 2; and which ring may be optionally substituted one or more times by C1-6 alkyl; or
R2 and R3 together form a 5-membered lactone ring (a) or a 6-membered lactone ring (b)

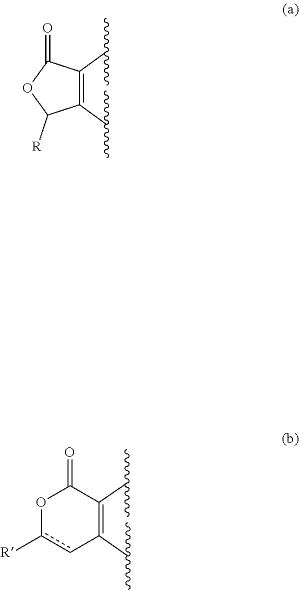

wherein [symbol] is an optional double bond and R′ is alkyl. [symbol]
Rc is selected from alkyl, alkenyl, alkynyl, aryl, arylalkyl, heteroaryl, heteroarylalkyl, carbocyclyl, carbocyclylalkyl, each of which may be optionally substituted;
or a pharmaceutically acceptable salt, solvate or prodrug thereof.

In some embodiments, A is optionally substituted aryl. In some embodiments, A is aryl substituted with —COOH, or a salt, ester or prodrug thereof. For example, A may be aryl substituted with —C0₂⁻NH₄⁺.

In some embodiments, R1 is CN.

In some embodiments, R₂ is acyl.

In some embodiments, the IRA inhibitor of the present invention has the structure:

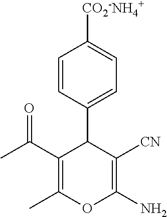

In some embodiments, the IRAP inhibitor of the present invention has a structure selected from the group consisting of:

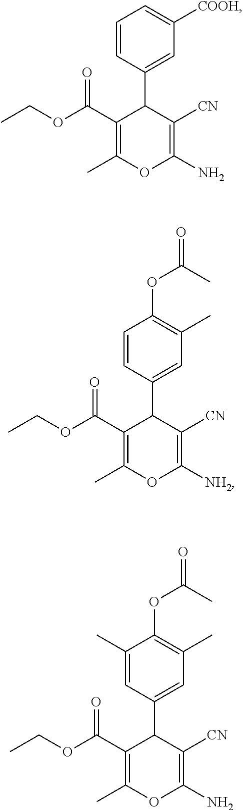 [SMILES: CCOC(=O)C1=C(C)OC(N)=C(C#N)C1c1cccc(C(=O)O)c1 ; CCOC(=O)C1=C(C)OC(N)=C(C#N)C1c1ccc(OC(C)=O)c(C)c1 ; CCOC(=O)C1=C(C)OC(N)=C(C#N)C1c1cc(C)c(OC(C)=O)c(C)c1]

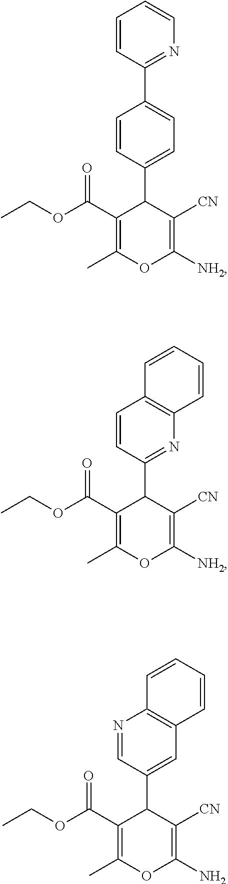 [SMILES: CCOC(=O)C1=C(C)OC(N)=C(C#N)C1c1ccc(-c2ccccn2)cc1 ; CCOC(=O)C1=C(C)OC(N)=C(C#N)C1c1ccc2ccccc2n1 ; CCOC(=O)C1=C(C)OC(N)=C(C#N)C1c1cnc2ccccc2c1]

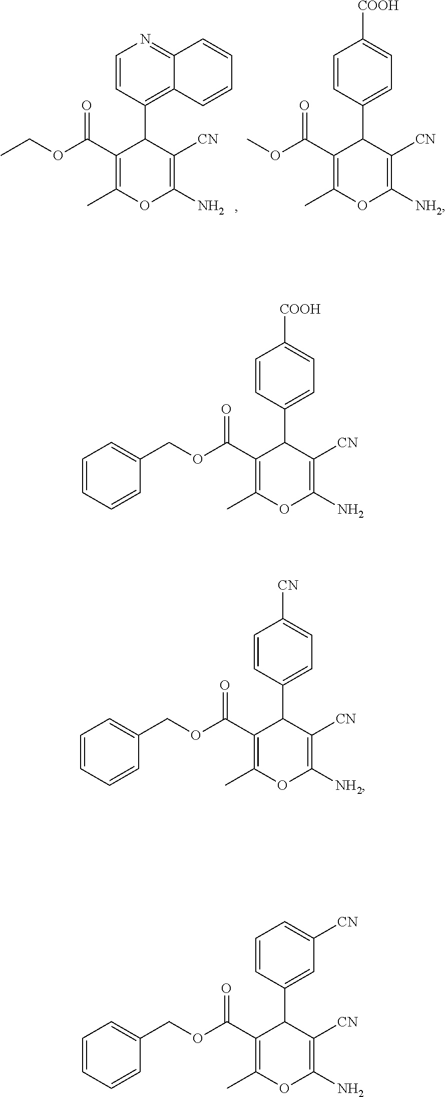

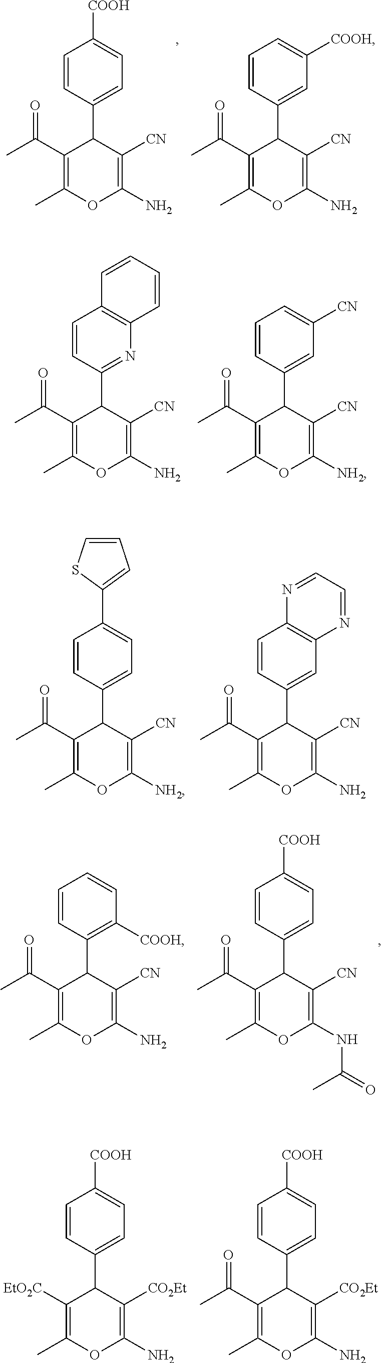

and/or a pharmaceutically acceptable salt, solvate or prodrug thereof.

In some embodiments, the IRAP inhibitor of the present invention has a structure according to Formula (III):

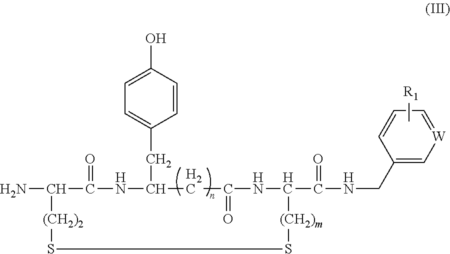

wherein
R1 is H or CH₂COOH; and
n is 0 or 1; and
m is 1 or 2; and
W is CH or N;
   or a pharmaceutically acceptable salt, solvate or prodrug thereof.

In some embodiment, the IRA inhibitor of the present invention has the structure:

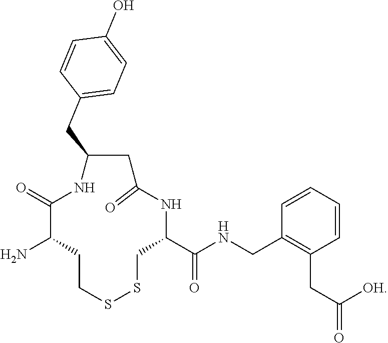 [SMILES: N[C@H]1CCSSC[C@@H](C(=O)NCc2ccccc2CC(=O)O)NC(=O)C[C@H](Cc2ccc(O)cc2)NC1=O]

In some embodiments, the IRAP inhibitor of the present invention has a structure according to the compound

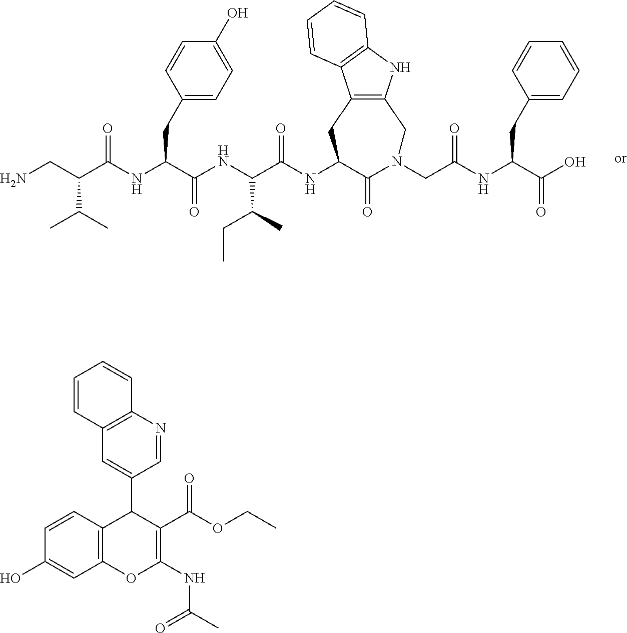

In some embodiments, the IRAP inhibitor of the present invention is (±)-Ethyl-2-acetamido-7-hydroxy-4-(pyridin-3-yl)-4H-chromene-3-carboxylate, also known as HFI-419, and that is described in Mountford, S. J., et al. 2014. J. Med. Chem. 57, 1368; Albiston, A. L., et al. 2011. Br. J. Pharmacol. 164, 37, Albiston, A. L., et al. 2010. Mol. Pharmacol. 78, 600; and Albiston, A. L., et al. 2008. FASEB J. 22, 4209. The compound has the formula of:

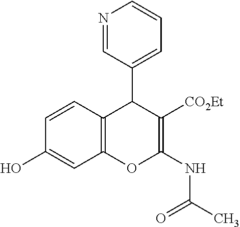 [SMILES: CCOC(=O)C1=C(NC(C)=O)Oc2cc(O)ccc2C1c1cccnc1]

As used herein, the term “alkyl” or “alk” denotes straight chain, or branched alkyl, preferably Ci₂o alkyl, e.g. C-M O or C1-6. Examples of straight chain and branched alkyl include methyl, ethyl, n-propyl, isopropyl, n-butyl, sec-butyl, f-butyl, n-pentyl, 1,2-dimethylpropyl, 1, 1-dimethyl-propyl, hexyl, 4-methylpentyl, 1-methylpentyl, 2-methylpentyl, 3-methylpentyl, 1, 1-dimethylbutyl, 2,2-dimethylbutyl, 3,3-dimethylbutyl, 1,2-dimethylbutyl, 1,3-dimethylbutyl, 1,2,2,-trimethylpropyl, 1, 1,2-trimethylpropyl, heptyl, 5-methylhexyl, 1-methylhexyl, 2,2-dimethylpentyl, 3,3-dimethylpentyl, 4,4-dimethylpentyl, 1,2-dimethylpentyl, 1,3-dimethylpentyl, 1,4-dimethyl-pentyl, 1,2,3-trimethylbutyl, 1, 1,2-trimethylbutyl, 1, 1,3-trimethylbutyl, octyl, 6-methylheptyl, 1-methylheptyl, 1, 1,3,3-tetramethylbutyl, nonyl, 1-2-, 3-, 4-, 5-, 6- or 7-methyl-octyl, 1-, 2-, 3-, 4- or 5-ethylheptyl, 1-, 2- or 3-propylhexyl, decyl, 1-, 2-, 3-, 4-, 5-, 6-, 7- and 8-methylnonyl, 1-, 2-, 3-, 4-, 5- or 6-ethyloctyl, 1-, 2-, 3- or 4-propylheptyl, undecyl, 1-, 2-, 3-, 4-, 5-, 6-, 7-, 8- or 9-methyldecyl, 1-, 2-, 3-, 4-, 5-, 6- or 7-ethylnonyl, 1-, 2-, 3-, 4- or 5-propylocytl, 1-, 2- or 3-butylheptyl, 1-pentylhexyl, dodecyl, 1-, 2-, 3-, 4-, 5-, 6-, 7-, 8-, 9- or 10-methylundecyl, 1-, 2-, 3-, 4-, 5-, 6-, 7- or 8-ethyldecyl, 1-, 2-, 3-, 4-, 5- or 6-propylnonyl, 1-, 2-, 3- or 4-butyloctyl, 1-2-pentylheptyl and the like. Where an alkyl group is referred to generally as “propyl”, butyl” etc, it will be understood that this can refer to any of straight or branched isomers where appropriate. An alkyl group may be optionally substituted by one or more optional substituents as herein defined.

The term “alkenyl” as used herein denotes groups formed from straight chain or branched hydrocarbon residues containing at least one carbon to carbon double bond including ethylenically mono-, di- or poly-unsaturated alkyl groups as previously defined, preferably 02-20 alkenyl (e.g. 02-10 or 02-Θ)—Examples of alkenyl include vinyl, allyl, 1-methylvinyl, butenyl, iso-butenyl, 3-methyl-2-butenyl, 1-pentenyl, 1-hexenyl, 3-hexenyl, 1-heptenyl, 3-heptenyl, 1-octenyl, 1-nonenyl, 2-nonenyl, 3-nonenyl, 1-decenyl, 3-decenyl, 1,3-butadienyl, 1-4,pentadienyl, 1,3-hexadienyl and 1,4-hexadienyl. An alkenyl group may be optionally substituted by one or more optional substituents as herein defined.

As used herein the term “alkynyl” denotes groups formed from straight chain or branched hydrocarbon residues containing at least one carbon-carbon triple bond including ethynically mono-, di- or poly-unsaturated alkyl groups as previously defined. Unless the number of carbon atoms is specified the term preferably refers to 02-20 alkynyl (e.g. C2-10 or C₂-6)—Examples include ethynyl, 1-propynyl, 2-propynyl, and butynyl isomers, and pentynyl isomers. An alkynyl group may be optionally substituted by one or more optional substituents as herein defined.

As used herein, terms written as “[group]oxy” refer to a particular group when linked by oxygen, for example, the terms “alkoxy”, “alkenoxy”, “alkynoxy”, “aryloxy” and “acyloxy” respectively denote alkyl, alkenyl, alkynyl, aryl and acyl groups as hereinbefore defined when linked by an oxygen atom. Terms written as “[group]thio” refer to a particular group when linked by sulfur, for example, the terms “alkylthio”, “alkenylthio”, alkynylthio” and “arylthio” respectively denote alkyl, alkenyl, alkynyl, aryl groups as hereinbefore defined when linked by a sulfur atom. Similarly, a term written as “[groupA]groupB” is intended to refer to a groupA when linked by a divalent form of groupB, for example, “hydroxyalkyl” is a hydroxy group when linked by an alkylene group.

As used herein, the term “halogen” (“halo”) denotes fluorine, chlorine, bromine or iodine (fluoro, chloro, bromo or iodo).

The term “aryl” (or “carboaryl)”, or the abbreviated form “ar” used in compound words such as “aralkyl”, denotes any of mono-, bi- or polcyclic, (including conjugated and fused) hydrocarbon ring systems containing an aromatic residue. Examples of aryl include phenyl, biphenyl, terphenyl, quaterphenyl, naphthyl, tetrahydronaphthyl (tetralinyl), anthracenyl, dihydroanthracenyl, benzanthracenyl, dibenzanthracenyl, phenanthrenyl, fluorenyl, pyrenyl, idenyl, isoindenyl, indanyl, azulenyl and chrysenyl. Particular examples of aryl include phenyl and naphthyl. An aryl group may be optionally substituted by one or more optional substituents as herein defined.

As used herein, the term “carbocyclyl” includes any of non-aromatic monocyclic, bicyclic and polycyclic, (including fused, bridged or conjugated) hydrocarbon residues, e.g. 03-20 (such as C-3-10, C3-8 or Cs-6). The rings may be saturated, for example cycloalkyl, or may possess one or more double bonds (cycloalkenyl) and/or one or more triple bonds (cycloalkynyl). Examples of particular carbocyclyl are monocyclic 5-6-membered or bicyclic 9-10 membered ring systems. Suitable examples include cyclopropyl, cyclobutyl, cyclopentyl, cyclohexyl, cycloheptyl, cyclooctyl, cyclononyl, cyclodecyl, cyclopentenyl, cyclohexenyl, cyclooctenyl, cyclopentadienyl, cyclohexadienyl, cyclooctatetraenyl and decalinyl. A carbocyclyl group may be optionally substituted by one or more optional substituents as herein defined. In particular, a monocarbocyclyl group may be substituted by a bridging group to form a bicyclic bridged group.

As used herein, the term “carbocyclyl” includes any of non-aromatic monocyclic, bicyclic and polycyclic, (including fused, bridged or conjugated) hydrocarbon residues, e.g. C₃₋₂o (such as C-3-10, C₃₋8 or Cs\hat{ }). The rings may be saturated, for example cycloalkyl, or may possess one or more double bonds (cycloalkenyl) and/or one or more triple bonds (cycloalkynyl). Examples of carbocyclyl include monocyclic 5-6-membered or bicyclic 9-10 membered ring systems.

Suitable examples include cyclopropyl, cyclobutyl, cyclopentyl, cyclohexyl, cycloheptyl, cyclooctyl, cyclononyl, cyclodecyl, cyclopentenyl, cyclohexenyl, cyclooctenyl, cyclopentadienyl, cyclohexadienyl, cyclooctatetraenyl and decalinyl. A carbocyclyl group may be optionally substituted by one or more optional substituents as herein defined. A monocarbocyclyl group may be substituted by a bridging group to form a bicyclic bridged group.

The term “heterocyclyl” when used alone or in compound words includes any of monocyclic, bicyclic or polycyclic, (including fuse, bridged or conjugated) hydrocarbon residues, such as C₃₋₂o (e.g. C₃₋₁₀ or C₃₋₈) wherein one or more carbon atoms are independently replaced by a heteroatom so as to provide a group containing a non-aromatic heteroatom containing ring. Suitable heteroatoms include, O, N, S, P and Se, particularly O, N and S. Where two or more carbon atoms are replaced, this may be by two or more of the same heteroatom or by different heteroatoms. The heterocyclyl group may be saturated or partially unsaturated, e.g. possess one or more double bonds. Particularly preferred heterocyclyl are monocyclic 5-6- and bicyclic 9-10-membered heterocyclyl. Examples of heterocyclyl groups may include azridinyl, oxiranyl, thiiranyl, azetidinyl, oxetanyl, thietanyl, 2H-pyrrolyl, pyrrolidinyl, 1-, 2- and 3-pyrrolinyl, piperidyl, piperazinyl, morpholinyl, indolinyl, imidazolidinyl, imidazolinyl, pyrazolidinyl, thiomorpholinyl, dioxanyl, tetrahydrofuranyl, tetrahydropyranyl, tetrahydropyrrolyl, tetrahydrothiophenyl (tetramethylene sulfide), pyrazolinyl, dioxalanyl, thiazolidinyl, isoxazolidinyl, dihydropyranyl, oxazinyl, thiazinyl, thiomorpholinyl, oxathianyl, dithianyl, trioxanyl, thiadiazinyl, dithiazinyl, trithianyl, azepinyl, oxepinyl, thiepinyl, indenyl, indanyl, 3H-indolyl, isoindolinyl, 4H-quinolazinyl, chromenyl, chromanyl, isochromanyl, benzoxazinyl (21-1-1,3, 21-1-1,4-, I H-2,3-, 41-1-3, 1-4H-1,4) pyranyl and dihydropyranyl. A heterocyclyl group may be optionally substituted by one or more optional substituents as defined herein.

As used herein, the term “heteroaryl” includes any of monocyclic, bicyclic, polycyclic, fused, bridged or conjugated hydrocarbon residues, wherein one or more carbon atoms are replaced by a heteroatom so as to provide a residue having at least one aromatic heteroatom-containing ring. Exemplary heteroaryl have 3-20 ring atoms, e.g. 3-10. Particularly preferred heteroaryl are 5-6 monocyclic and 9-10 membered bicyclic ring systems. Suitable heteroatoms include, O, N, S, P and Se, particularly O, N and S. Where two or more carbon atoms are replaced, this may be by two or more of the same heteroatom or by different heteroatoms. Suitable examples of heteroaryl groups may include pyridyl, pyrrolyl, thienyl, imidazolyl, furanyl, benzothienyl, isobenzothienyl, benzofuranyl, isobenzofuranyl, indolyl, isoindolyl, pyrazolyl, pyrazinyl, pyrimidinyl, pyridazinyl, indolizinyl, quinolyl, isoquinolyl, phthalazinyl, 1,5-naphthyridinyl, quinozalinyl, quinazolinyl, quinolinyl, oxazolyl, thiazolyl, isothiazolyl, isoxazolyl, triazolyl, oxadialzolyl, oxatriazolyl, triazinyl, tetrazolyl and furazanyl. A heteroaryl group may be optionally substituted by one or more optional substituents as defined herein.

As used herein, the term “acyl” either alone or in compound words denotes a group containing the moiety C=0. In some embodiments acyl does not include a carboxylic acid, ester or amide. Acyl includes C(0)-Z, wherein Z is hydrogen or an alkyl, aryl, heteroaryl, carbocyclyl, heterocyclyl, arylalkyl, heteroarylalkyl, carbocyclylalkyl, or heterocyclylalkyl residue. Examples of acyl include formyl, straight chain or branched alkanoyl (e.g. Ci-2o) such as, acetyl, propanoyl, butanoyl, 2-methylpropanoyl, pentanoyl, 2,2-dimethylpropanoyl, hexanoyl, heptanoyl, octanoyl, nonanoyl, decanoyl, undecanoyl, dodecanoyl, tridecanoyl, tetradecanoyl, pentadecanoyl, hexadecanoyl, heptadecanoyl, octadecanoyl, nonadecanoyl and icosanoyl; cycloalkylcarbonyl such as cyclopropylcarbonyl cyclobutylcarbonyl, cyclopentylcarbonyl and cyclohexylcarbonyl; aroyl such as benzoyl, toluoyl and naphthoyl; aralkanoyl such as phenylalkanoyl (e.g. phenylacetyl, phenylpropanoyl, phenylbutanoyl, phenylisobutylyl, phenylpentanoyl and phenylhexanoyl) and naphthylalkanoyl (e.g. naphthylacetyl, naphthylpropanoyl and naphthylbutanoyl]; aralkenoyl such as phenylalkenoyl (e.g. phenylpropenoyl, phenylbutenoyl, phenylmethacryloyl, phenylpentenoyl and phenylhexenoyl and naphthylalkenoyl (e.g. naphthylpropenoyl, naphthylbutenoyl and naphthylpentenoyl); aryloxyalkanoyi such as phenoxyacetyl and phenoxypropionyl; arylthiocarbamoyi such as phenylthiocarbamoyl; arylglyoxyloyl such as phenylglyoxyloyl and naphthylglyoxyloyl; arylsulfonyl such as phenylsulfonyl and napthylsulfonyl; heterocycliccarbonyl; heterocyclicalkanoyl such as thienylacetyl, thienylpropanoyl, thienylbutanoyl, thienylpentanoyl, thienylhexanoyl, thiazolylacetyl, thiadiazolylacetyl and tetrazolylacetyl; heterocyclicalkenoyl such as heterocyclicpropenoyl, heterocyclicbutenoyl, heterocyclicpentenoyl and heterocyclichexenoyl; and heterocyclicglyoxyloyl such as thiazolyglyoxyloyl and thienylglyoxyloyl. The R and Z residues may be optionally substituted as described herein. In this specification “optionally substituted” is taken to mean that a group may be unsubstituted or further substituted or fused (so as to form a condensed bi- or polycyclic group) with one, two, three or more of organic and inorganic groups, including those selected from: alkyl, alkenyl, alkynyl, carbocyclyl, aryl, heterocyclyl, heteroaryl, acyl, aralkyl, alkylaryl, alkylheterocyclyl, alkylheteroaryl, alkylcarbocyclyl, halo, haloalkyl, haloalkenyl, haloalkynyl, haloaryl, halocarbocyclyl, haloheterocyclyl, haloheteroaryl, haloacyl, haloaryalkyl, hydroxy, hydroxyalkyl, hydroxyalkenyl, hydroxyalkynyl, hydroxycarbocyclyl, hydroxyaryl, hydroxyheterocyclyl, hydroxyheteroaryl, hydroxyacyl, hydroxyaralkyl, alkoxyalkyl, alkoxyalkenyl, alkoxyalkynyl, alkoxycarbocyclyl, alkoxyaryl, alkoxyheterocyclyl, alkoxyheteroaryl, alkoxyacyl, alkoxyaralkyl, alkoxy, alkenyloxy, alkynyloxy, aryloxy, carbocyclyloxy, aralkyloxy, heteroaryloxy, heterocyclyloxy, acyloxy, haloalkoxy, haloalkenyloxy, haloalkynyloxy, haloaryloxy, halocarbocyclyloxy, haloaralkyloxy, haloheteroaryloxy, haloheterocyclyloxy, haloacyloxy, nitro, nitroalkyl, nitroalkenyl, nitroalkynyl, nitroaryl, nitroheterocyclyl, nitroheteroaryl, nitrocarbocyclyl, nitroacyl, nitroaralkyi, amino (NH₂), alkylamino, dialkylamino, alkenylamino, alkynylamino, arylamino, diarylamino, aralkylamino, diaralkylamino, acylamino, diacylamino, heterocyclamino, heteroarylamino, carboxy, carboxyester, amido, alkylsulphonyloxy, arylsulphenyloxy, alkylsulphenyl, arylsulphenyl, thio, alkylthio, alkenylthio, alkynylthio, arylthio, aralkylthio, carbocyclylthio, heterocyclylthio, heteroarylthio, acylthio, sulfoxide, sulfonyl, sulfonamido, aminoalkyl, aminoalkenyl, aminoalkynyl, aminocarbocyclyl, aminoaryl, aminoheterocyclyl, aminoheteroaryl, aminoacyl, aminoaralkyl, thioalkyl, thioalkenyl, thioalkynyl, thiocarbocyclyl, thioaryl, thioheterocyclyl, thioheteroaryl, thioacyl, thioaralkyl, carboxyalkyl, carboxyalkenyl, carboxyalkynyl, carboxycarbocyclyl, carboxyaryl, carboxyheterocyclyl, carboxyheteroaryl, carboxyacyl, carboxyaralkyl, carboxyesteralkyl, carboxyesteralkenyl, carboxyesteralkynyl, carboxyestercarbocyclyl, carboxyesteraryl, carboxyesterheterocyclyl, carboxyesterheteroaryl, carboxyesteracyl, carboxyesteraralkyl, amidoalkyl, amidoalkenyl, amidoalkynyl, amidocarbocyclyl, amidoaryl, amidoheterocyclyl, amidoheteroaryl, amidoacyl, amidoaralkyl, formylalkyl, formylalkenyl, formylalkynyl, formylcarbocyclyl, formylaryl, formylheterocyclyl, formylheteroaryl, formylacyl, formylaralkyl, acylalkyl, acylalkenyl, acylalkynyl, acylcarbocyclyl, acylaryl, acylheterocyclyl, acylheteroaryl, acylacyl, acylaralkyl, sulfoxidealkyl, sulfoxidealkenyl, sulfoxidealkynyl, sulfoxidecarbocyclyl, sulfoxidearyl, sulfoxideheterocyclyl, sulfoxideheteroaryl, sulfoxideacyl, sulfoxidearalkyl, sulfonylalkyl, sulfonylalkenyl, sulfonylalkynyl, sulfonylcarbocyclyl, sulfonylaryl, sulfonylheterocyclyl, sulfonylheteroaryl, sulfonylacyl, sulfonylaralkyl, sulfonamidoalkyl, sulfonamidoalkenyl, sulfonamidoalkynyl, sulfonamidocarbocyclyl, sulfonamidoaryl, sulfonamidoheterocyclyl, sulfonamidoheteroaryl, sulfonamidoacyl, sulfonamidoaralkyl, nitroalkyl, nitroalkenyl, nitroalkynyl, nitrocarbocyclyl, nitroaryl, nitroheterocyclyl, nitroheteroaryl, nitroacyl, nitroaralkyi, cyano, sulfate, sulfonate, phosphonate and phosphate groups. Optional substitution may also be taken to refer to where a CH₂ group in a chain or ring is replaced by a carbonyl group (C═O) or a thiocarbonyl group (C═S), where 2 adjacent or non-adjacent carbon atoms (e.g. 1,2- or 1,3) are substituted by one end each of a —O— (CH₂)S—O— or —NR_{X}—(CH₂)S—NR_{X}-group, wherein s is 1 or 2 and each R^{x} is independently H or Cₕalkl!, and where 2 adjacent or non-adjacent atoms, independently selected from C and N, are substituted by one end each of a C₁₋₅alkylene or C₂₋₅alkenylene group (so as to form a bridged group). Exemplary optional substituents include those selected from: alkyl, (e.g. CHalkyl such as methyl, ethyl, propyl, butyl), cycloalkyl (e.g. C3-6cycloalkyl, such as cyclopropyl, cyclobutyl, cyclopentyl or cyclohexyl), hydroxyalkyl (e.g. hydroxyC1-6 alkyl, such as hydroxymethyl, hydroxyethyl, hydroxypropyl), alkoxyalkyl (e.g. C₁₋₆alkoxyC1-6 alkyl, such as methoxymethyl, methoxyethyl, methoxypropyl, ethoxymethyl, ethoxyethyl, ethoxypropyl), alkoxy (e.g. C₁₋₆ alkoxy, such as methoxy, ethoxy, propoxy, butoxy), alkoxyalkoxy (e.g. C₁₋₆alkoxyC₁₋₆alkoxy, such as methoxymethoxy, methoxyethoxy, methoxypropoxy, ethoxymethoxy, ethoxyethoxy, ethoxypropoxy, propoxymethoxy, propoxyethoxy, propoxypropoxy) cycloalkoxy (e.g. cyclopropoxy, cyclobutoxy, cyclopentoxyl, cyclohexyloxy), halo, haloalkyl(e.g. haloC1-6 alkyl, such as chloromethyl, difluoromethyl, trifluoromethyl, trichloromethyl, tribromomethyl), haloalkoxy (e.g. halo C₁₋₆ alkoxy), hydroxy, thio (—SH), sulfonyl, sulfonamide, phenyl (which itself may be further substituted e.g., by one or more C₁₋₆ alkyl, halo, hydroxy, hydroxy C₁₋₆ alkyl, C₁₋₆ alkoxy, C₁₋₆ alkoxy C₁₋₆ alkyl, C₁₋₆ alkoxy C₁₋₆ alkoxy, halo C₁₋₆ alkyl, halo C₁₋₆ alkoxy, cyano, nitro, OC(O) C₁₋₆ alkyl, NH₂, NH C₁₋₆ alkyl, NHC(O) C₁₋₆ alkyl and NC1-6 alkylC1-6 alkyl), benzyl (wherein benzyl itself may be further substituted e.g., by one or more of CHalkyl, halo, hydroxy, hydroxy C₁₋₆ alkyl, C₁₋₆ alkoxy, C₁₋₆ alkoxy C₁₋₆ alkyl, C1-6 alkoxyC1-6 alkoxy, haloC1-6alkyl, haloC₁₋₆alkoxy, cyano, nitro, OC(0)C₁₋₆alkyl, NH₂, NHC₁₋₆alkyl, NHC(O)C₁₋₆alkyl and NC1-6 alkylC1-6 alkyl), phenoxy (wherein phenyl itself may be further substituted e.g., by one or more of CHalkyl, halo, hydroxy, hydroxyC1-6 alkyl, C1-6 alkoxy, C1-6 alkoxyCi-₆alkyl, C1-6 alkoxyC1-6 alkoxy, haloC1-6 alkyl, haloC1-6 alkoxy, cyano, nitro, OC(0)C1-6 alkyl, NH₂, NHC1-6 alkyl, NHC(O)C1-6 alkyl and NC1-6 alkylC1-6 alkyl), benzyloxy (wherein benzyl itself may be further substituted e.g., by one or more of C1-6 alkyl, halo, hydroxy, hydroxyC1-6 alkyl, C1-6 alkoxy, C1-6 alkoxyC1-6 alkyl, C1-6 alkoxyC1-6 alkoxy, haloi₋₆alkyl, haloC1-6 alkoxy, cyano, nitro, OC(0)C1-6 alkyl, NH₂, NHC1-6 alkyl, NHC(O)C1-6 alkyl and NC1-6 alkylC1-6 alkyl), NH₂, alkylamino (e.g. —NHC1-6 alkyl, such as methylamino, ethylamino, propylamino etc), dialkylamino (e.g. —NH(C1-6 alkyl)₂, such as dimethylamino, diethylamino, dipropylamino), acylamino (e.g. —NHC(0)C1-6 alkyl, such as —NHC(0)CH₃), phenylamino (i.e. —NHphenyl, wherein phenyl itself may be further substituted e.g., by one or more of CHalkyl, halo, hydroxy, hydroxyC1-6 alkyl, hydroxyC1-6 alkoxy C1-6 alkoxy, C1-6 alkoxyC1-6 alkyl, C1-6 alkoxyC1-6 alkoxy, haloC1-6 alkyl, haloC1-6 alkoxy, cyano, nitro, OC(0)C₁₋₆alkyl, NH₂, NHC₁₋₆alkyl, NHC(O)C₁₋₆alkyl and NC₁₋₆alkylC₁₋₆alkyl), nitro, cyano, formyl, —C(0)-alkyl (e.g. —C(0)C1-6 alkyl, such as acetyl), 0-C(0)-alkyl (e.g. —OC(0)C1-6 alkyl, such as acetyloxy), benzoyl (wherein benzyl itself may be further substituted e.g., by one or more of CHalkyl, halo, hydroxy, hydroxyC1-6 alkyl, C1-6 alkoxy, C1-6 alkoxyC1-6 alkyl, C1-6 alkoxyC1-6 alkoxy, haloC1-6 alkyl, haloC1-6 alkoxy, cyano, nitro, OC(0)C1-6 alkyl, NH2, NHC1-6 alkyl, NHC(O)C1-6 alkyl and NC1-6 alkylC1-6 alkyl), benzoyloxy (wherein benzyl itself may be further substituted e.g., by one or more of C1-6 alkyl, halo, hydroxy, hydroxyC1-6 alkyl, C1-6 alkoxy, C1-6 alkoxyC1-6 alkyl, C1-6 alkoxyC1-6 alkoxy, haloC-i-₆alkyl, haloC1-6 alkoxy, cyano, nitro, OC(0)C1-6 alkyl, NH₂, NHC1-6 alkyl, NHC(0)C1-6 alkyl and NC1-6 alkylC1-6 alkyl), CO₂H, C0₂alkyl (e.g. C0₂C1-6 alkyl such as methyl ester, ethyl ester, propyl ester, butyl ester), CO₂phenyl (wherein phenyl itself may be further substituted e.g., by one or more of C1-6 alkyl, halo, hydroxy, hydroxyC1-6 alkyl, C1-6 alkoxy, C1-6 alkoxyC1-6 alkyl, C1-6 alkoxyC1-6 alkoxy, haloC1-6 alkyl, haloC1-6 alkoxy, cyano, nitro, OC(0)C1-6 alkyl, NH2, NHC1-6 alkyl, NHC(0)C1-6 alkyl and NC1-6 alkylC1-6 alkyl), C0₂benzyl (wherein benzyl itself may be further substituted e.g., by one or more of CHalkyl, halo, hydroxy, hydroxyC1-6 alkyl, C1-6 alkoxy, C1-6 alkoxyC1-6 alkyl, C1-6 alkoxyC1-6 alkoxy, haloC-i.₆alkyl, haloC1-6 alkoxy, cyano, nitro, OC(0)C1-6 alkyl, NH₂, NHC1-6 alkyl, NHC(O)C1-6 alkyl and NC1-6 alkylC1-6 alkyl), CONH₂, C(0)NHphenyl (wherein phenyl itself may be further substituted e.g., by one or more of CHalkyl, halo, hydroxy, hydroxyC1-6 alkyl, C1-6 alkoxy, C1-6 alkoxyC1-6 alkyl, C1-6 alkoxyC1-6 alkoxy, haloC1-6 alkyl, haloC1-6 alkoxy, cyano, nitro, OC(0)C₁₋₆alkyl, NH₂, NHC₁₋₆alkyl, NHC(0)C₁₋₆alkyl and NC₁₋₆alkylC₁₋₆alkyl), C(0)NHbenzyl (wherein benzyl itself may be further substituted e.g., by one or more of C1-6 alkyl, halo, hydroxy, hydroxyC1-6 alkyl, C1-6 alkoxy, C1-6 alkoxyC1-6 alkyl, C1-6 alkoxyCi-₆alkoxy, haloC1-6 alkyl, haloC1-6 alkoxy, cyano, nitro, OC(0)C1-6 alkyl, NH2, NHC1-6 alkyl, NHC(O)C₁₋₆alkyl and NC₁₋₆alkylC₁₋₆alkyl), C(0)NHalkyl (e.g. C(0)NHC₁₋₆ alkyl such as methyl amide, ethyl amide, propyl amide, butyl amide) C(0)Ndialkyl (e.g. C(0)N(C-|.₆alkyl)₂) aminoalkyl (e.g., HNC1-6 alkyl-, C1-6 alkylHN—C1-6 alkyl- and (C1-6 alkyl)₂N—C1-6 alkyl-), thioalkyl (e.g., HSC1-6 alkyl-), carboxyalkyl (e.g., H0₂CC1-6 alkyl-), carboxyesteralkyl (e.g., C1-6 alkyl0₂CC1-6 alkyl-), amidoalkyl (e.g., H₂N(0)CC1-6 alkyl-H(C1-6 alkyl)N(0)CCi-₆alkyl-), formylalkyl (e.g., OHCC₁₋₆alkyl-), acylalkyl (e.g., C₁₋₆alkyl(0)CC₁₋₆alkyl-), nitroalkyl (e.g., 0₂NC1-6 alkyl-), replacement of CH₂ with C=0, replacement of CH₂ with C═S, substitution of 2 adjacent or non-adjacent carbon atoms (e.g. 1,2 or 1,3) by one end each of a -0-(CH₂)ₛ-0- or —NR′—(CH₂)s-NR′-group, wherein s is 1 or 2 and each R′ is independently H or CHalkyl, and substitution of 2 adjacent or non-adjacent atoms, independently selected from C and N, by a C₂₋₅alkylene or C₂₋₅alkenylene group.

The term “sulfoxide”, either alone or in a compound word, refers to a group —S(0)R wherein R is selected from hydrogen, alkyl, alkenyl, alkynyl, aryl, heteroaryl, heterocyclyl, carbocyclyl, and aralkyl. Examples of R include hydrogen, C-i₋₂oalkyl, phenyl and benzyl. The term “sulfonyl”, either alone or in a compound word, refers to a group S(0)₂—R, wherein R is selected from hydrogen, alkyl, alkenyl, alkynyl, aryl, heteroaryl, heterocyclyl, carbocyclyl, acyl, and aralkyl. Examples of R include hydrogen, C-i₋₂₀alkyl, phenyl and benzyl.

As used herein, the term “sulfonamide”, or “sulfonamyl” of “sulfonamido”, either alone or in a compound word, refers to a group S(0)₂NRR wherein each R is independently selected from hydrogen, alkyl, alkenyl, alkynyl, aryl, heteroaryl, heterocyclyl, carbocyclyl, acyl, and aralkyl. Examples of R include hydrogen, C-i-₂oalkyl, phenyl and benzyl. In an embodiment at least one R is hydrogen. In another form, both R are hydrogen.

As used herein, the term “sulfamate”, either alone or in a compound word, refers to a group —OS(0)₂NRR wherein each R is independently selected from hydrogen, alkyl, aryl, heteroaryl, heterocyclyl, carbocyclyl, acyl, and aralkyl. Examples of R include hydrogen, Ci₋₂oalkyl, phenyl and benzyl. In an embodiment at least one R is hydrogen. In another form, both R are hydrogen.

As used herein, the term “sulfamide”, either alone or in a compound word, refers to a group —NRS(O)₂NRR wherein each R is independently selected from hydrogen, alkyl, aryl, heteroaryl, heterocyclyl, carbocyclyl, acyl, and aralkyl. Examples of R include hydrogen, Ci₋₂₀alkyl, phenyl and benzyl. In an embodiment at least one R is hydrogen. In another form, both R are hydrogen. As used herein, the term “sulfate” group refers to a group —OS(0)₂OR wherein each R is independently selected from hydrogen, alkyl, alkenyl, alkynyl, aryl, heteroaryl, heterocyclyl, carbocyclyl, acyl, and aralkyl. Examples of R include hydrogen, Ci₋₂oalkyl, phenyl and benzyl. As used herein, the term “sulfonate” refers to a group S0₃R wherein each R is independently selected from hydrogen, alkyl, alkenyl, alkynyl, aryl, heteroaryl, heterocyclyl, carbocyclyl, acyl, and aralkyl. Examples of R include hydrogen, C-i₋₂oalkyl, phenyl and benzyl.

As used herein, the term “thio” is intended to include groups of the formula “—SR” wherein R can be hydrogen (thiol), alkyl, alkenyl, alkynyl, aryl, carbocyclyl, heteroaryl, heterocyclyl, aralkyl, and acyl. Examples of R include hydrogen, C-i₋₂oalkyl, phenyl and benzyl.

As used herein, the term “amino” is used here in its broadest sense as understood in the art and includes groups of the formula —NR_{A}RB wherein R_{A} and R_{B} may be independently selected from hydrogen, hydroxy alkyl, alkoxyalkyl, aryl, heteroaryl, carbocyclyl, heterocyclyl, arylalkyl, heteroarylalkyl, carbocyclylalkyl, heterocyclylalkyl, acyl and amido, each of which may be optionally substituted as described herein. R_{A} and R_{B}, together with the nitrogen to which they are attached, may also form a monocyclic, or fused polycyclic ring system e.g. a 3-10-membered ring, particularly, 5-6 and 9-10-membered systems. Examples of “amino” include —NH₂, —NHalkyl (e.g. —NHC-i₋₂oalkyl), —NHalkoxyalkyl, —NHaryl (e.g. —NHphenyl), —NHaralkyl (e.g. —NHbenzyl), —NHacyl (e.g. —NHC(O)C₁₋₂₀alkyl, —NHC(O)phenyl), —NHamido, (e.g. NHC(0)NHiC₁₋₆alkyl, NHC(O)NH phenyl), —Ndialkyl (wherein each alkyl, for example Ci-2o, may be the same or different) and 5 or 6 membered rings, optionally containing one or more same or different heteroatoms (e.g. O, N and S). Reference to groups written as “[group]amino” is intended to reflect the nature of the R_{A} and R_{B} groups. For example, “alkylamino” refers to —NR_{A}R_{B} where one of R_{A} or R_{B} is alkyl. “Dialkylamino” refers to —NR_{A}R_{B} where R_{A} and R_{B} are each (independently) an alkyl group.

As used herein, the term “amido” is used here in its broadest sense as understood in the art and includes groups having the formula C(0)NR_{A}R_{B}, wherein R_{A} and R_{B} are as defined as above. Examples of amido include C(0)NH₂, C(0)NHalkyl (e.g. Ci₋₂₀alkyl), C(0)NHaryl (e.g. C(O)NHphenyl), C(0)NHaralkyl (e.g. C(O)NHbenzyl), C(0)NHacyl (e.g. C(O)NHC(O)Ci₋₂₀alkyl, C(0)NHC(0)phenyl), C(0)Nalkylalkyl (wherein each alkyl, for example Ci₋₂₀, may be the same or different) and 5 or 6 membered rings, optionally containing one or more same or different heteroatoms (e.g. O, N and S).

As used herein, the term “carboxy ester” is used here in its broadest sense as understood in the art and includes groups having the formula —C0₂R, wherein R may be selected from groups including alkyl, alkenyl, alkynyl, aryl, carbocyclyl, heteroaryl, heterocyclyl, arylalkyl, heteroarylalkyl, carbocyclylalkyl, heterocyclylalkyl, aralkenyl, heteroarylalkenyl, carbocyclylalkenyl, heterocyclylalkenyl, aralkynyl, heteroarylalkynyl, carbocyclylalkynyl, heterocyclylalkynyl, and acyl, each of which may be optionally substituted. Some examples of carboxy ester include —CO₂C-i₋₂₀alkyl, —C0₂aryl (e.g. —C0₂phenyl), —CO₂arCi₋₂₀alkyl (e.g. —C0₂ benzyl).

As used herein, the term “phosphonate” refers to a group —P(0)(OR₂) wherein R is independently selected from hydrogen, alkyl, aryl, heteroaryl, heterocyclyl, carbocyclyl, acyl, and aralkyl. Examples of R include hydrogen, Ci₋₂oalkyl, phenyl and benzyl.

As used herein, the term “phosphate” refers to a group —OP(O)(OR)₂ wherein R is independently selected from hydrogen, alkyl, aryl, heteroaryl, heterocyclyl, carbocyclyl, acyl, and aralkyl. Examples of R include hydrogen, Ci₋₂oalkyl, phenyl and benzyl.

Carboxyclic isosteres are groups which can exhibit the same or similar properties as a carboxylic group. Some examples of carboxylic acid isosteres include: —SO3H, —S0₂NHR, —P0₂R₂, —CN, —P0₂R₂, —OH, —OR, —SH, —SR, —NHCOR, —NR₂, —CONR₂, —CONH(O)R, —CONHNHSO₂R, —COHNSO₂R and —CONR—CN, where R is selected from H, alkyl (such as d-6 alkyl), phenyl and benzyl. Other carboxylic acid isosteres include carbocyclic and heterocyclic groups such as:

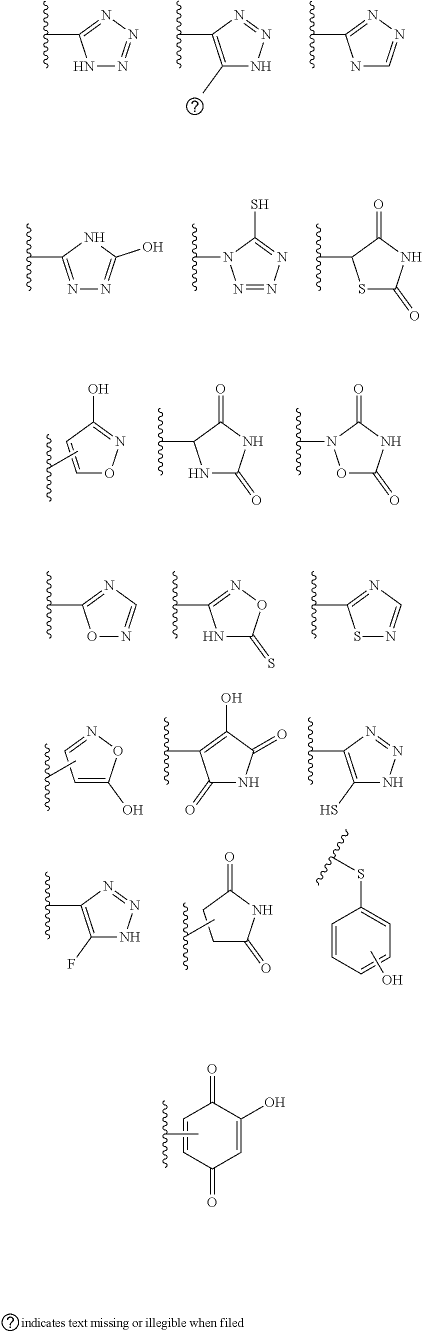

co

A used herein, the term “pharmaceutically-acceptable salts” refers to those salts which, within the scope of sound medical judgement, are suitable for use in contact with the tissues of humans and animals without undue toxicity, irritation, allergic response and the like, and are commensurate with a reasonable benefit/risk ratio. Pharmaceutically acceptable salts are well known in the art. Examples of such inorganic acids are hydrochloric, hydrobromic, hydroiodic, nitric, carbonic, sulfuric, and phosphoric acid. Appropriate organic acids may be selected from aliphatic, cycloaliphatic, aromatic, heterocyclic carboxylic and sulfonic classes of organic acids, examples of which are formic, acetic, propionic, succinic, glycolic, gluconic, lactic, malic, tartaric, citric, ascorbic, glucoronic, fumaric, maleic, pyruvic, alkyl sulfonic, arylsulfonic, aspartic, glutamic, benzoic, anthranilic, mesylic, salicylic, p-hydroxybenzoic, phenylacetic, mandelic, ambonic, pamoic, pantothenic, sulfanilic, cyclohexylaminosulfonic, stearic, algenic, O-hydroxybutyric, galactaric, and galacturonic acids. Suitable pharmaceutically acceptable base addition salts of the compounds of the present invention include metallic salts made from lithium, sodium, potassium, magnesium, calcium, aluminium, and zinc, and organic salts made from organic bases such as choline, diethanolamine, morpholine. Alternatively, organic salts made from N,N′-dibenzylethylenediamine, chloroprocaine, choline, diethanolamine, ethylenediamine, meglumine (N methylglucamine), procaine, ammonium salts, quaternary salts such as tetramethylammonium salt, amino acid addition salts such as salts with glycine and arginine. For example, alkali metal salts (K, Na) and alkaline earth metal salts (Ca, Mg) may be used if deemed appropriate for the structure, but again any pharmaceutically acceptable, non-toxic salt may be used where appropriate. The Na- and Ca-salts are preferred. Pharmaceutically acceptable solvates, including hydrates, of such compounds and such salts are also intended to be included within the scope of this invention.

In some embodiments, the IRAP inhibitor leads to the destabilization and/or degradation of IRAP. In some embodiments, the IRAP inhibitor is a compound that targets the degradation of IRAP. For instance, the compound can be a Proteolysis Targeting Chimera (PROTAC). PROTACs are heterobifunctional compounds composed of a target protein-binding ligand and an E3 ubiquitin ligase ligand, and induce proteasome-mediated degradation of selected proteins via their recruitment to E3 ubiquitin ligase and subsequent ubiquitination. These drug-like molecules offer the possibility of temporal control over protein expression. Such compounds are capable of inducing the inactivation of a protein of interest upon addition to cells or administration to an animal or human, and could be useful for degrading pathogenic or oncogenic proteins (Crews C, Chemistry & Biology, 2010, 17(6):551-555; Schnnekloth J S Jr., Chembiochem, 2005, 6(1):40-46).

In some embodiments, the IRAP inhibitor is an inhibitor of IRAP expression. An “inhibitor of expression” refers to a natural or synthetic compound that has a biological effect to inhibit the expression of a gene. In a preferred embodiment of the invention, said inhibitor of gene expression is a siRNA, an antisense oligonucleotide or a ribozyme. For example, anti-sense oligonucleotides, including anti-sense RNA molecules and anti-sense DNA molecules, would act to directly block the translation of IRAP mRNA by binding thereto and thus preventing protein translation or increasing mRNA degradation, thus decreasing the level of IRAP, and thus activity, in a cell. For example, antisense oligonucleotides of at least about 15 bases and complementary to unique regions of the mRNA transcript sequence encoding IRAP can be synthesized, e.g., by conventional phosphodiester techniques. Methods for using antisense techniques for specifically inhibiting gene expression of genes whose sequence is known are well known in the art (e.g. see U.S. Pat. Nos. 6,566,135; 6,566,131; 6,365,354; 6,410,323; 6,107,091; 6,046,321; and 5,981,732). Small inhibitory RNAs (siRNAs) can also function as inhibitors of expression for use in the present invention. IRAP gene expression can be reduced by contacting a subject or cell with a small double stranded RNA (dsRNA), or a vector or construct causing the production of a small double stranded RNA, such that IRAP gene expression is specifically inhibited (i.e. RNA interference or RNAi). Antisense oligonucleotides, siRNAs, shRNAs and ribozymes of the invention may be delivered in vivo alone or in association with a vector. In its broadest sense, a “vector” is any vehicle capable of facilitating the transfer of the antisense oligonucleotide, siRNA, shRNA or ribozyme nucleic acid to the cells and typically cells expressing IRAP. Typically, the vector transports the nucleic acid to cells with reduced degradation relative to the extent of degradation that would result in the absence of the vector. In general, the vectors useful in the invention include, but are not limited to, plasmids, phagemids, viruses, other vehicles derived from viral or bacterial sources that have been manipulated by the insertion or incorporation of the antisense oligonucleotide, siRNA, shRNA or ribozyme nucleic acid sequences. Viral vectors are a preferred type of vector and include, but are not limited to nucleic acid sequences from the following viruses: retrovirus, such as moloney murine leukemia virus, harvey murine sarcoma virus, murine mammary tumor virus, and rous sarcoma virus; adenovirus, adeno-associated virus; SV40-type viruses; polyoma viruses; Epstein-Barr viruses; papilloma viruses; herpes virus; vaccinia virus; polio virus; and RNA virus such as a retrovirus. One can readily employ other vectors not named but known to the art.

According to the invention, the IRAP inhibitor is administered to the patient in a therapeutically effective amount. By a “therapeutically effective amount” is meant a sufficient amount of the active ingredient for treating or reducing the symptoms at reasonable benefit/risk ratio applicable to any medical treatment. It will be understood that the total daily usage of the compounds and compositions of the present invention will be decided by the attending physician within the scope of sound medical judgment. The specific therapeutically effective dose level for any particular subject will depend upon a variety of factors including the disorder being treated and the severity of the disorder; activity of the specific compound employed; the specific composition employed, the age, body weight, general health, sex and diet of the patient; the time of administration, route of administration, and rate of excretion of the specific compound employed; the duration of the treatment; drugs used in combination with the active ingredients; and like factors well known in the medical arts. For example, it is well within the skill of the art to start doses of the compound at levels lower than those required to achieve the desired therapeutic effect and to gradually increase the dosage until the desired effect is achieved. However, the daily dosage of the products may be varied over a wide range from 0.01 to 1,000 mg per adult per day. Typically, the compositions contain 0.01, 0.05, 0.1, 0.5, 1.0, 2.5, 5.0, 10.0, 15.0, 25.0, 50.0, 100, 250 and 500 mg of the active ingredient for the symptomatic adjustment of the dosage to the patient to be treated. A medicament typically contains from about 0.01 mg to about 500 mg of the active ingredient, typically from 1 mg to about 100 mg of the active ingredient. An effective amount of the drug is ordinarily supplied at a dosage level from 0.0002 mg/kg to about 20 mg/kg of body weight per day, especially from about 0.001 mg/kg to 7 mg/kg of body weight per day.

Typically the IRAP inhibitor is combined with pharmaceutically acceptable excipients, and optionally sustained-release matrices, such as biodegradable polymers, to form pharmaceutical compositions. The term “pharmaceutically” or “pharmaceutically acceptable” refers to molecular entities and compositions that do not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type. The carrier can also be a solvent or dispersion medium containing, for example, water, ethanol, polyol (for example, glycerol, propylene glycol, and liquid polyethylene glycol, and the like), suitable mixtures thereof, and vegetables oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminium monostearate and gelatin. In the pharmaceutical compositions of the present invention, the active ingredients of the invention can be administered in a unit administration form, as a mixture with conventional pharmaceutical supports. Suitable unit administration forms comprise oral-route forms such as tablets, gel capsules, powders, granules and oral suspensions or solutions, sublingual and buccal administration forms, aerosols, implants, subcutaneous, transdermal, topical, intraperitoneal, intramuscular, intravenous, subdermal, transdermal, intrathecal and intranasal administration forms and rectal administration forms.

The invention will be further illustrated by the following figures and examples. However, these examples and figures should not be interpreted in any way as limiting the scope of the present invention.

### FIGURES

FIG. 1. IRAP endosomes are required for pro-inflammatory cytokine secretion in mast cells.

(A) Peritoneal mast cells were stimulated with ionomycin/PMA for 18 h and secreted cytokines were quantified by ELISA in the culture supernatant. Graphs represent mean±SEM of three or more experiments.

(B) Peritoneal mast cells were stimulated for 4 h with ionomycin/PMA and TAPI-I, and plasma membrane-bound TNF-α on live cells was detected via flow cytometry. Graphs represent mean±SEM of four experiments.

*p<0.05,**p<0.01, ***p<0.001

FIG. 2. IRAP endosomes are required for inflammatory cytokine secretion in vivo.

(A-E) IRAPwt and ko mice (A-C) or mast-cell deficient Wsh mice reconstituted with IRAPwt and ko BMMC (D,E) were challenged on one ear with 30 mg/ml arachidonic acid while the control ear was left untreated. Cytokine concentrations were quantified in ear tissue homogenates and normalized to total protein concentration.

(F) Arthritis score of IRAPwt and ko mice 8 days after CAIA induction. Graphs show pooled data from two independent experiments.

(G) Kidney injury score of cisplatin-treated IRAPwt and ko mice.

(H) Plasma TNF-α concentration of cisplatin-treated IRAPwt and ko mice 24 h after cisplatin injection from one out of three experiments.

(I) Injury scores of paraffin kidney sections of cisplatin-treated IRAPwt or ko BMMC-reconstituted kit-Wsh/sh mice from three independent experiments.

*p<0.05,**p<0.01, ***p<0.001

FIG. 3. IRAP inhibitor HFI-419 blocks cytokine secretion via destabilization of IRAP and VAMP3+ endosomes

IRAP wt mice were injected i.v. with 6 μg HI-419 or vehicle 24 h and 15 min prior to ear challenge. Cytokine concentrations were quantified in ear tissue homogenates and normalized to total protein concentration. Graph shows one out two similar experiments.

### EXAMPLE

### Methods

### Study Design

We used IRAPko and wt mice to study the role of IRAP endosomes in mast cell functions. To this aim, we investigated bone-marrow-derived mast cells and peritoneal mast cells in vitro with regards to degranulation and cytokine secretion. We used the TNF-dependent inflammation models of autoantibody-induced arthritis and cisplatin-mediated kidney injury in wt versus IRAPko mouse as well as in mast cell-deficient kit-W^{sh/sh} mice that had been reconstituted with IRAPko or wt mast cells. To unravel the underlying cell biological mechanism we performed IF and image stream co-localization experiments with relevant endosomal markers and analyzed the Golgi export kinetics of TNF. Experiments with the chemical IRAP inhibitor HI-419 complete the study.

### Reagents and Antibodies

The following antibodies were used in this study. Mouse monoclonal TRAP antibody clone 3E, rabbit monoclonal anti-TRAP XP clone D7C5, rabbit anti-EEA1 (all Cell Signaling); rat anti-mouse lysosome-associated membrane protein (LAMP)1 clone 1D4B, mouse monoclonal anti-STX6, mouse monoclonal anti-GM130 (BD Pharmingen); rabbit polyclonal anti-STX6 (ProteinTech Group, Chicago, IL, United States); mouse monoclonal anti-Stx4 clone QQ-17 (Santa Cruz); rabbit polyclonal anti-TNF (abcam 34674 for confocal imaging and imaging flow cytometry), PE-PerCP5.5 anti-mouse TNF clone MP6-XT22 (eBiosciences for FACS); rat anti-IL-6 and rat anti-IL-10 (eBiosciences); rabbit polyclonal anti-VAMP3 (abcam 2102); rabbit polyclonal anti-VAMP8 (novus); goat anti-serotonin (abcam 66047), rabbit polyclonal anti-Rab14 (Sigma Aldrich). All secondary reagents were Alexa-coupled highly cross-adsorbed antibodies from Molecular Probes (Life technologies). Alexa647-transferrin was from Life technologies. IL-3 and SCF (premium grade) were purchased from Milteny Biotec.

Murine cytokine detection Duoset enzyme-linked immunosorbent assay (ELISA) kits were from R&D Systems (mIL-6, mTNF) or from Biolegends (mIL-10). Easysep™ anti-mouse CD117 positive selection kit was from Stemcell. The TNF-alpha-converting enzyme (TACE) inhibitor TAPI-1, ionomycin, PMA, HFI-419, dynasore, GDC-0941 were all from Calbiochem. p-Nitrophenyl-N-acetyl-β-D-glucosaminide (pNAG) was from Sigma. The IRAP inhibitors 4u and 11b were a gift from E.Stratikos (Demokritos Research Center Athens).

### Mice

Previously described IRAP^{-/-} mice on an Sv129 background obtained from S. Keller were back-crossed up to 10 times to C57BL/6 mice obtained from Janvier (St. Quentin-Fallavier, France). Control wt mice were C57BL/6 mice bred in our facility or C57BL/6 mice purchased from Charles River. Kit-W^{sh/sh} were purchased from Jackson Laboratories (strain #30764). Animal experimentation was conducted in agreement with the guidelines of local authorities, approved by the Comité d'Éthique pour l'Expdrimentation Animale at Paris Descartes.

### Mast Cell Isolation and Culture

Murine bone marrow-derived mast cells (BMMCs) were produced in vitro by culturing cells extruded from large bones for 4 to 6 weeks in complete medium [Iscove's modified Dulbecco's medium (IMDM) complemented with 10% fetal calf serum (FCS), 25 mM

HEPES (pH 7.4), 2 mM glutamine, 100 U/ml penicillin, 100 g/ml streptomycin, 50 μM β-mercaptoethanol, 1% non-essential amino acids and 1 mM sodium pyruvate] supplemented with 10 ng/ml IL-3.

Every 5-7 days, medium was replaced. All cell cultures were grown at 37° C. in a humidified atmosphere with 5% CO2. BMMC differentiation as verified by staining with CD117 and FcεR antibodies after 4 weeks was more than 98%. Mouse peritoneal-derived mast cells (PCMCs) were obtained by peritoneal lavage with 5 mL ice-cold PBS/0.1% BSA and cultured in complete medium (see above) supplemented with 10 ng/ml IL-3 and 10 ng/ml SCF.

Non-adherent cells including mast cells were separated from adherent macrophages after 3 h of culture. Cultured cells were enriched for mast cells (>90%) after 7 days of culture. For use after shorter culture times, mast cells were purified via anti-CD117 beads (StemCell).

### Mast Cell Reconstitution of Kit-W^{sh/sh} Mice

BMMC from wt and IRAPko mice were cultured for 4 weeks in the presence of murine IL-3 and murine SCF as described above. 5×10⁶ BMMC were injected i.v. in kit-W^{sh/sh} mice and allowed for 8 to 12 weeks for reconstitution before functional experiments. Reconstituted mice yielding less than 50 nM histamine per μg total protein in untreated ear tissue homogenates were considered as unsuccessfully reconstituted and excluded from the analysis.

### Flow Cytometric Assays

For TNF-α surface staining, PCMCs were stimulated with 1 μM ionomycin/10 nM PMA or 100 ng/ml LPS at 37° C. for 4 h in the presence of TAPI-1, washed with ice-cold PBS, and incubated at 4° C. with Fcblock (Miltenyi) followed by fluorochrome-conjugated CD117, FcεRI and TNF-α antibodies diluted in PBS-1% BSA. Intracellular staining of cytokines, IRAP and VAMP3 was performed using the BD intracellular staining kit and suitable species-specific fluorescent secondary antibodies (Life technologies).

For degranulation experiments, PCMCs were stimulated with 1 μM ionomycin/10 nM PMA or 48/80 for 30 min at 37° C., placed on ice and surface-stained with AlexaFluor488 anti-LAMP1. BD Canto™ and Gallios flow cytometers were used for cell analysis.

### Mouse Ear Challenge

Twenty microliters arachidonic acid (AA) (30 mg/ml in acetone) were applied to the inner and outer surface of one mouse ear, whereas the other ear was left untreated.

One hour after AA application, mice were sacrificed and ears were collected. The ear biopsies were dissociated using the pre-set “Protein” protocol of gentleMACS™ Octo Dissociator (Milteniy Biotec) in 800 μl ice-cold homogenization buffer [(PBS containing 0.4M NaCl, 0.05% Tween-20, 10 mM EDTA and protease inhibitor cocktail complete (Roche)]. The homogenates were cleared by 10 min centrifugation at 5000×g, and the total protein concentration determined in a BCA assay. Histamine or cytokines in the supernatant were quantified as described below.

### Cytokine and Histamine Measurements

PCMCs were stimulated with 1 uM ionomycin/10 nM PMA or 100 ng/ml LPS at 37° C. for 6 h for cytokine secretion or with ionomycin/PMA or 10 ug/ml 48/80 for 30 min for histamine measurement.

Supernatants were collected and histamine was quantified using the Histamine Dynamic HTRF kit (Cisbio). TNF-α, IL-6, or IL-10 were quantified using specific cytokine ELISA kits. Kits were used according to the manufacturer's instructions.

### Beta-Hexosaminidase Release

PCMCs were stimulated with 1 μM ionomycin/10 nM PMA or 10 ug/ml 48/80 for 30 min in Tyrode's buffer. Following stimulation, cell suspensions were centrifuged, placed on ice and supernatants were collected. The cell pellets of unstimulated cells were lysed with 0.5% Triton X-100 to determine the maximal enzymatic activity of 0-hexosaminidase. Twenty-five microliters of supernatant or the lysate volume corresponding to 5×10³ cells were incubated with 50 μl of a 1.3 mg/ml p-Nitrophenyl-N-acetyl-β-D-glucosaminide (pNAG) solution in 50 mM citrate buffer pH 4.5 at 37° C. for 90 min. The reaction was stopped with 150 ul of 0.2 M glycine buffer pH 10.7 and absorbance was read at 405 nm. The percentage of degranulation was expressed as the ratio of absorbance of a given supernatant to the absorbance measured in the lysates of unstimulated cells.

### Cisplatin-Induced Kidney Injury Model

Mice were injected intraperitoneally with 10 mg/kg cisplatin. Blood samples for measurement of plasma TNF-α levels were taken at 24 h after cisplatin injection. Mice were sacrificed at 96 h, and kidneys were processed for histological analysis as described in the histology section below. Tubular injury was independently scored in a blinded manner by three investigators.

### Collagen-Antibody Induced Arthritis Model

Mice were injected intravenously with 4 mg/mouse antibody cocktail to collagen II (Chondrex, Inc.) on day 0, followed by an intraperitoneal LPS injection (25 ug/mouse) on day 3. Severity of arthritis was evaluated on day 8 according to a qualitative scoring system as followed: 0—normal, 1—mild but definite redness of the ankle or wrist, or apparent redness and swelling limited to individual digits, 2—moderate redness and swelling of ankle or wrist, 3—severe redness and swelling of the entire paw including digits, 4—maximally inflamed limb involving multiple joints. Mice were sacrificed and hind legs were collected, and processed for histological analysis as described below.

### Histology

Mouse ears, kidneys or hind legs were collected and fixed in 10% formalin for 24 h. Hind legs were decalcified in 1M EDTA solution for 1 week. After paraffin embedding, 4 μm sagittal (legs), transversal (ears) or coronal sections (kidneys) were cut and stained with hematoxylin/eosin or periodic acid Schiff stain as indicated. Tissue sections were imaged using a Leica DM2000 microscope equipped with a MC160HD camera using 5× and 20× objectives.

### Confocal Microscopy

BMMCs were seeded on IBIDI poly-lysin-coated microscopy chambers in complete medium containing IL-3 at 37° C. in a humidified atmosphere with 5% CO2 for 16 h, stimulated as indicated, washed in PBS and fixed in PBS-4% PFA for 15 min at room temperature. Permeabilization, blocking, washes and antibody incubation were performed in PBS-0.1% saponin/0.2% BSA at 18° C. Image acquisition was performed on a Zeiss LSM700 with an 63× oil-immersion objective. Images were analyzed and assembled using FIJI with the FigureJ plug-in.

### Imaging Flow Cytometry

One million BMMCs were stimulated as indicated, fixed with 4% PFA for 10 min, permeabilized with permeabilization buffer (Invitrogen) and stained for indicated markers for 30 min at RT, followed by a washing step in permeabilization buffer and secondary staining with fluorescently labeled antibodies for 30 min at RT. Cells were washed, resuspended in PBS-2% FCS. Image acquisition was performed at 60× magnification using an ImageStream XMkII multispectral imaging flow cytometer (Amnis Corp., Seattle, USA), and acquired images were analyzed with the IDEAS software (version 6.2; Amnis Corp.). For SNARE protein analysis, a Stx4+ mask was defined and the mean pixel intensity of VAMP8 or VAMP3 was measured inside the mask. In the Golgi export assay, the Golgi mask was defined by GM130 staining and TNF mean pixel intensity quantified within the Golgi mask.

### Statistical Analysis

Values are expressed as mean±SEM, unless otherwise specified. Statistical significance between two groups was analyzed using the unpaired t-test with Welch's correction, or one-sample t-test where replicates were expressed as percentage of a control group. P values are indicated as: *p<0.05; **p<0.01; ***p<0.001; ****p<0.0001, ns=non-significant. GraphPad Prism version 9.0 was used to perform the statistical analysis.

### Results

### IRAP Endosomes are Dispensable for Secretory Granule Exocytosis

Aiming to shed light on the role of RAP endosomes in mast cell exocytic trafficking pathways, we first colocalized IRAP with different endosomal markers of early and GSV-like endosomes (data not shown). Like in dendritic cells, IRAP colocalized well with the early endosomal markers EEA1 and endocytized transferrin in mast cells, as well as with the GSV markers Rab14 and Stx6 involved in Golgi-to-endosome trafficking, confirming a high level of conservation of the IRAP-related vesicular trafficking machinery amongst different cell types. Interestingly, in activated cells, IRAP strongly colocalized with the granule-contained monoamine serotonin at the plasma membrane (data not shown), similar to the observation with regards to histamine in the initial study³¹, which prompted us to re-examine the role of IRAP in mast cell degranulation.

Physiologically, ligation of mast cell surface receptors, including crosslinking of FcεR through IgE and cognate antigen, activates signaling cascades most of which converge to Ca²⁺ release from intracellular stores. If and how secretion of pre-stored granules versus de novo synthesized mediators upon Ca²⁺ signaling is regulated, is unknown.

As we aimed to analyze the potential involvement of IRAP endosomes in exocytosis separately from its hypothetical “upstream” role in the FcεR-related signaling cascade³¹, we exclusively used FcεR-independent activation of mast cells throughout our study.

We stimulated peritoneal mast cells with ionomycin/PMA or with the GPCR-dependent compound 48/80⁴⁶, and measured degranulation either as the release of the major granule component beta-hexosaminidase into the culture supernatant (data not shown), or exocytosis of the lysosomal marker LAMP-1 in a flow cytometry assay (data not shown). Degranulation was significantly increased in IRAP knock-out (IRAPko) cells upon 48/80 activation while the ionomycin/PMA stimulation led to strong degranulation responses without significant differences between IRAP-expressing and -deficient cells. As saturation effects may hide differences upon stimulation by ionomycin/PMA, we turned to an in vitro assay for the measurement of histamine release. This FRET-based technique is quantitative over a large range of histamine concentrations and detected significantly increased degranulation in the absence of IRAP for both types of stimulation (data not shown).

The exocytosis of secretory granules depends on the SNARE VAMP8. We observed no or poor colocalization of IRAP with VAMP8 and preformed TNF-α stored in secretory granules in resting mast cells (data not shown). Prior to release, granule-bound VAMP8 assembles with the t-SNAREs Stx4 and SNAP23 at the plasma membrane or in intracellular degranulation channels (Moon et al., 2014). Also during the degranulation process we failed to observe any colocalization of IRAP with VAMP8 (data not shown) confirming the localization to distinct endosomal compartments.

However, we wondered if the increased release of granule content in IRAPko mast cells was reflected by an increased complex formation of VAMP8 with Stx4 upon activation. We therefore quantified colocalization of VAMP8-positive granules with Stx4 by imaging flow cytometry after stimulation with ionomycin/PMA. As expected, we observed more VAMP8 colocalization with Stx4 in IRAPko than in wild-type (wt) mast cells (data not shown). Next, we sought to assess degranulation in vivo. To this end, we challenged IRAP wt and ko mice on one ear for degranulation with arachidonic acid, while the other ear was left untreated. Arachidonic acid induces degranulation and cytokine production in mast cells through the prostaglandin EP3 receptor⁴⁷. In line with our in vitro results, we detected significantly more histamine in crude homogenates of stimulated IRAPko mouse ears than in wt ears (data not shown). This was not due to different mast cell densities in tissues of wt compared to IRAPko mice (data not shown).

To confirm the mast cell-specificity of this test, we reconstituted mast cell-deficient kit-W^{sh/sh} mice (Wsh) with bone marrow-derived mast cells (BMMC) from wt or IRAPko donor mice and challenged them along with non-reconstituted Wsh mice. As expected, no histamine was detected in the ear homogenates from the mast cell-deficient, non-reconstituted Wsh mice, while histamine secretion was increased in the challenged ears in Wsh mice reconstituted with IRAPko BMMC compared to wt BMMC (data not shown). It is important to note that this assay does not discriminate the origin of the detected histamine from intracellular stores versus extracellular locations after degranulation. It is, however, likely that the histamine epitopes recognized in the antibody-based detection assay are more exposed after exocytosis, explaining the net increase of detectable histamine in challenged ears containing IRAPko mast cells, while smaller quantities of released molecules in wt ears might not be detectable with this protocol due to a strong background signal generated by histamine from intracellular stores.

In conclusion, we show that IRAP endosomes are dispensable for the VAMP8-dependent pathway of regulated secretion in mast cells, and moreover, in their absence, degranulation is increased in vitro and in vivo.

### Constitutive Secretion of Cytokines Relies on IRAP Endosomes in Mast Cells

Next to the regulated secretion of stored granule contents, mast cells produce and secrete de novo synthesized cytokines via the constitutive secretion pathway. Although both species of secreted vesicles originate from the Golgi and engage with Stx4 and SNAP23 for docking and fusion at the plasma membrane, they follow distinct post-Golgi trafficking routes. Thus, while regulated secretion depends on VAMP8, de novo synthesized cytokines in the constitutive pathway in murine mast cells stain with VAMP3¹⁹. We observed that IRAP endosomes colocalized well with VAMP3 in mast cells (data not shown). VAMP3 is associated with Golgi trafficking to and from the recycling compartment and has been implicated in TNF-α secretion in macrophages²¹. In mast cells, TNF-α is stored in limited amounts in secretory granules, and de novo produced and secreted via the constitute pathway in the late phase of activation. TNF-α is transported throughout the cell as a transmembrane pro-cytokine. Release of soluble TNF-α into the extracellular space requires the activity of the TNF-α-cleaving enzyme TACE. In the presence of the TACE inhibitor TAPI-I TNF-α accumulates at the surface of activated cells starting from 1 h of activation, where it colocalized strongly with IRAP (data not shown). We therefore hypothesized that TRAP might be involved in the constitutive secretion pathway of cytokines in mast cells.

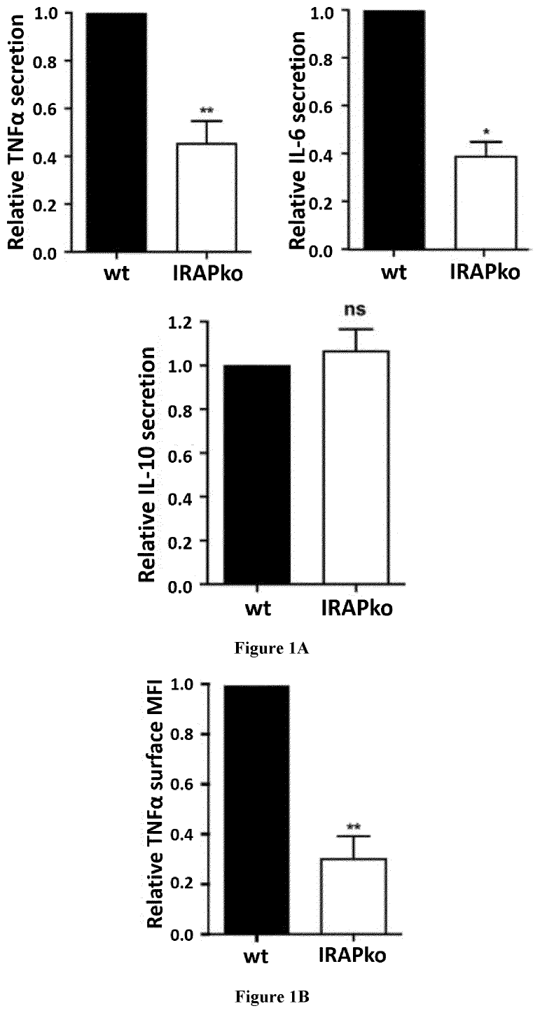

Indeed, TNF-α and IL-6 secretion was reduced about 50% in IRAPko compared to wt peritoneal mast cells after ionomycin/PMA stimulation as determined via ELISA (FIG. 1A) or TAPI-I treatment and TNF-α surface staining followed by flow cytometry analysis (FIG. 1B). Of note, secretion of the regulatory cytokine IL-10 was not affected by the absence of IRAP (FIG. 1A).

In order to verify that the observed secretion defect was due to a trafficking defect in IRAPko cells rather than a diminished synthesis rate, we compared intracellular cytokine levels after 4 h of activation under inhibition of Golgi/post-Golgi trafficking with brefeldin A. As we failed to detect any significant differences in the quantities of intracellularly produced cytokines between IRAP wt and ko mast cells under these conditions (data not shown), we concluded that the absence of IRAP endosomes produces a trafficking defect of de novo synthesized IL-6 and TNF within or beyond the Golgi. This defect also translates into reduced quantities of VAMP3-bearing vesicles colocalizing with Stx4 at the plasma membrane in IRAPko cells detectable by confocal imaging (data not shown) and imaging flow cytometry (data not shown).

Macrophages increase VAMP3 expression under LPS stimulation, possibly to cope with the need for more transport machinery upon increased cytokine synthesis²¹. To test if the same was true for IRAP expression, we stimulated mast cells for different periods of time with LPS and measured IRAP expression by intracellular flow cytometry. Indeed, IRAP was induced over time (data not shown), compatible with a role in pro-inflammatory cytokine trafficking.

### IRAP Endosomes are Required for TNF-α Secretion In Vivo

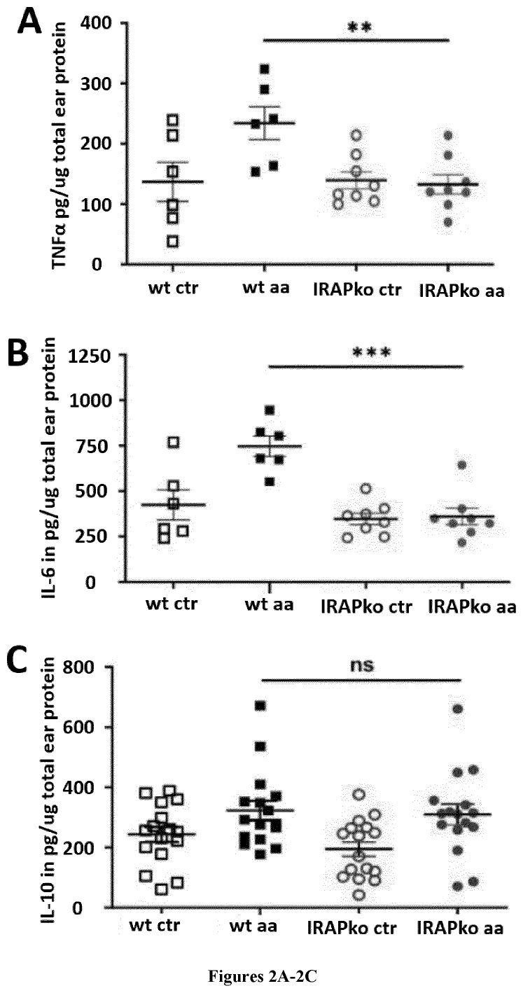

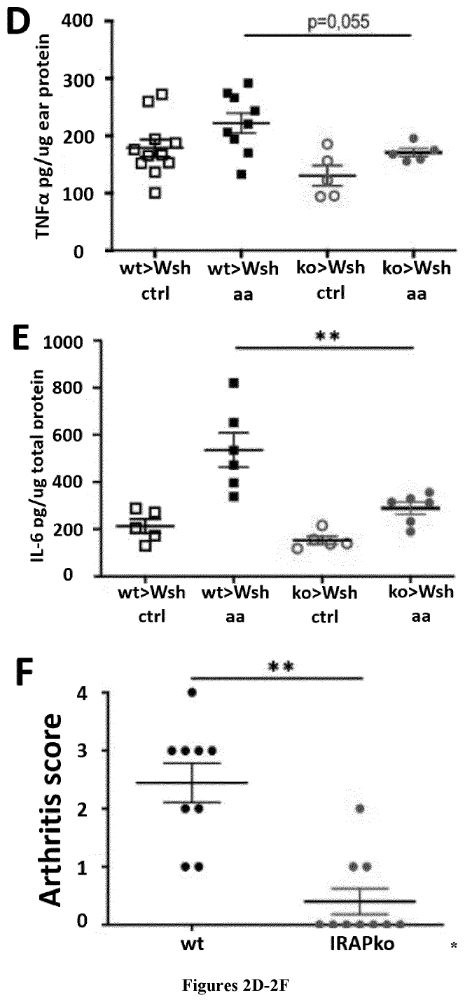

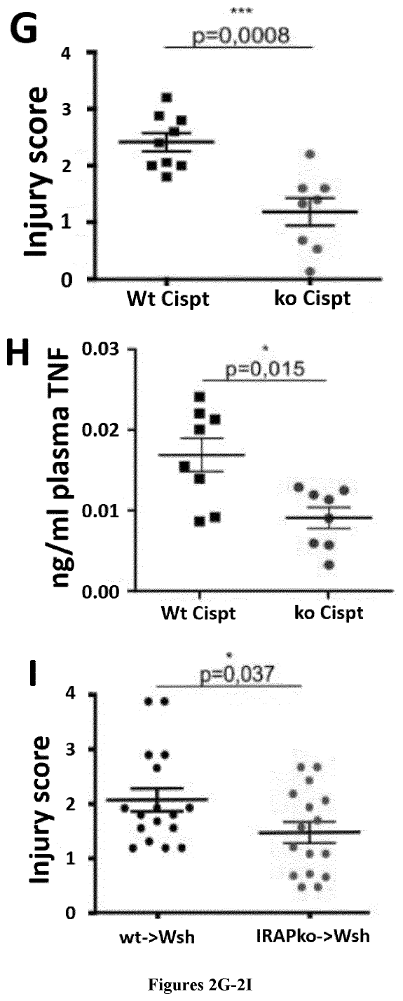

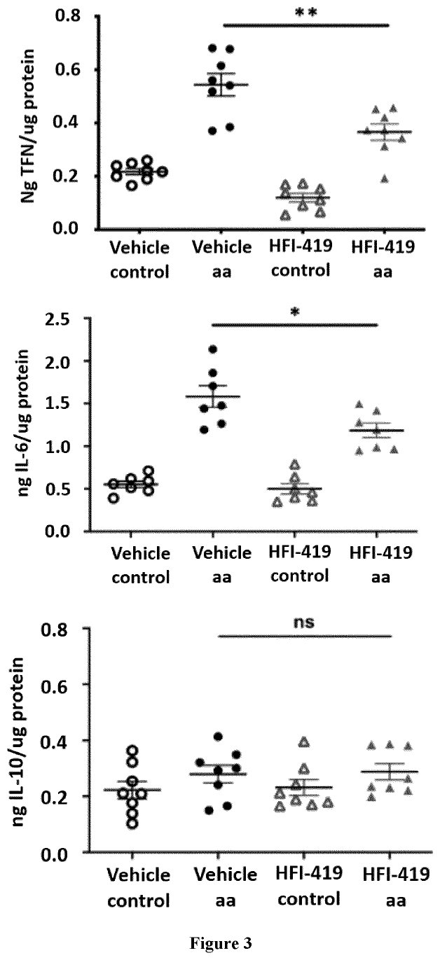

To quantify cytokine secretion by mast cells in vivo, we performed the mouse ear challenge experiment from above. While increased TNF-α and IL-6 levels were detected in wt ears within 45 min after the challenge, no cytokine release was observed in the ears of IRAPko animals (FIGS. 2A and B). Confirming our in vitro results, IL-10 secretion was not affected by the lack of IRAP endosomes in vivo (FIG. 2C). Searching to monitor the mast-cell contribution to the observed effects, we repeated the test in Wsh mice that had been reconstituted with wt or IRAPko BMMC. Wsh mice reconstituted with IRAPko mast cells showed defects in TNF-α and IL-6 secretion indicating that the cytokines measured in this experimental setting could indeed be attributed to mast cells (FIGS. 2D and E).

The role of TNF-α in the pathogenesis of collagen-induced arthritis (CAIA) is well documented^{48,49} To examine the relevance of IRAP endosomes for TNF-α secretion in this disease model, we challenged wt and IRAPko mice with an arthritogenic collagen-directed antibody cocktail. Eight days after arthritis induction, wt mice presented signs of joint inflammation marked by intense redness, swelling of paws and joints and difficulties to walk, while the majority of IRAPko mice showed no or only mild symptoms (FIG. 2F). Histological analyses of the knee (data not shown) and ankles (data not shown) revealed joint swelling accompanied by strong infiltration of inflammatory cells into the synovial space and bone erosion in wt animals. IRAPko mice showed less or no infiltrations, less swelling and no bone damage. We conclude that IRAP is required for the strong inflammatory disease phenotype observed in wt mice.

As the role of mast cells in this model has been somewhat questioned by the fact that Kit-W^{v/v} but not Wsh mice were protected from CAIA^{13,50} presumably due to differences in their megakaryocyte populations⁵¹, we turned to a cisplatin-induced kidney inflammation model previously reported to depend on mast cell-derived TNF-α as an alternative approach¹². Cisplatin is an efficient and widely employed cytostatic agent for cancer therapy the tolerance for which, however, is limited by the frequent adverse effect of acute kidney injury. We hypothesized that the TNF-α-dependent kidney injury after cisplatin administration that is characterized by tubular apoptosis, necrosis and inflammation, would be attenuated in IRAPko mice. To verify this, we histologically analyzed the kidneys of IRAP wt and ko mice 96 h after peritoneal cisplatin injection. HE (data not shown) and PAS (data not shown) staining of paraffin-embedded kidney samples revealed visibly reduced tubular damage in IRAPko animals and translated into significantly lower injury scores that were determined independently in a blinded evaluation by three different experimenters (FIG. 2G). These observations were consistent with significantly reduced TNF-α plasma levels in cisplatin-treated IRAPko mice (FIG. 2H). Cisplatin-induced inflammation and nephrotoxicity are mediated via TLR4⁵². In order to exclude the possibility that different TLR4 expression levels of wt versus IRAPko cells were at the origin of the observed effects, we confirmed comparable TLR4 surface expression of wt and IRAPko mast cells (data not shown).

To evaluate the contribution of mast cells to these effects, we administered cisplatin to wt or IRAPko BMMC-reconstituted Wsh mice. Scoring of the histological injury level indicated that the mean kidney damage of mice reconstituted with IRAPko mast cells was reduced as compared to mice reconstituted with wt mast cells (FIG. 2I), although the difference was less pronounced than between wt versus ubiquitously IRAPko mice. The involvement of other TNF-α-producing cell types in the tested cisplatin model might explain the limited differences in the experiments with mast cell-reconstituted mice. We therefore addressed TNF-α secretion in peritoneal macrophages using confocal imaging and the TAPI-based flow cytometry assay described above. IRAP colocalized strongly with TNF-α in ionomycin-activated macrophages (data not shown). Furthermore, IRAPko macrophages showed diminished TNF surface staining after 4 hours of activation by ionomycin/PMA or LPS (data not shown). We conclude that also macrophages depend on IRAP endosomes for the efficient secretion of TNF-α via the constitutive pathway.

Taken together, IRAPko mast cells, and likely other immune cell types, secrete less TNF-α in vivo leading to milder phenotypes in TNF-α-dependent disease models.

### IRAP is Required for Golgi Export of TNF-α Transport Vesicles

We next sought to unravel at which step the exocytic cytokine trafficking was impaired in the absence of IRAP. To this end we analyzed Stx6 colocalization with VAMP3 in activated mast cells. Stx6 decorates IRAP vesicles in different cell types and is present on TNF-α carriers after budding from the Golgi in macrophages^{21,22}. While Stx6 colocalized well with VAMP3 at the plasma membrane in activated mast cells, significantly less Stx6 was detected in the VAMP3-stained areas in IRAPko cells due to overall reduced peripheral Stx6 staining (data not shown). Total VAMP3 levels are also reduced in IRAPko mast cells (data not shown).

These observations prompted us to inquire if TNF-α carriers required IRAP for budding from the Golgi. We therefore adapted a previously published Golgi export assay⁵³. LPS-pre-activated cells were incubated at 20° C. for 3 h to enrich cytokines in the Golgi. Subsequent temperature shift to 37° C. re-activates budding of exocytic transport vesicles from the Golgi allowing for analysis of Golgi export kinetics of cytokines in the constitutive pathway.

After 3 h at 20° C., TNF-α colocalized strongly with the Golgi marker GM130 in both wt and IRAP ko cells (data not shown), indicating successful inhibition of Golgi export under these conditions. Re-activation of exocytic trafficking resulted in progressive export of TNF-α from the Golgi in wt cells, while in IRAPko cells, a net accumulation was observed over the first 30 min, indicating that the translation rate exceeded the export rate in these cells (data not shown). At 50 min, TRAP expressing cells had largely emptied the Golgi of TNF-α, while in IRAPko cells, colocalization of TNF-α and GM130 persisted (data not shown).

Considering that the cytosolic retention pool of TRAP vesicles, upon activation, has been proposed to translocate to the plasma membrane without passing through the Golgi^{38,54}, intersection with TNF-α carriers is difficult to envisage. However, under prolonged activating signaling, IRAP is reinternalized and retrieved to the Golgi from sorting endosomes via retromer action³⁶. We therefore wondered if endocytosis inhibition changed the subcellular localization of IRAP and ultimately TNF-α secretion. Indeed, in the presence of the dynamin inhibitor dynasore IRAP showed a strong plasma membrane staining (data not shown) after three hours of LPS activation indicating efficient inhibition of IRAP re-internalization. Consistently, both dynasore as well as the PI3K I inhibitor GDC-0941 were able to reduce TNF-α secretion specifically in wt cells (data not shown). These results suggest that IRAP internalization is a prerequisite for normal TNF-α trafficking (data not shown).

### IRAP Inhibition by HFI-419 Destabilizes IRAP Endosomes

Considering the aminopeptidase function of TRAP, we wondered if its catalytic activity was required for efficient cytokine secretion. To test this, we treated mast cells with the IRAP inhibitors HFI-419, 4u 55 and 22b (a gift from E. Stratikos) for 24 h prior to ionomycin/PMA activation. Although all three inhibitors showed a tendency to inhibit IRAP-dependent cytokine secretion in vitro, only HFI-419 mediated significant inhibition (data not shown) and was therefore selected for further in vivo studies.

Vehicle or inhibitor at a dose of 1 μmol/kg was administered intravenously at 24 h and 15 min prior to the ear challenge. HFI-419-treated animals secreted significantly less TNF-α and IL-6 than vehicle-treated animals, while IL-10 secretion was unaffected, indicating that the availability of the catalytic domain of IRAP was directly or indirectly required for trafficking of these pro-inflammatory cytokines (FIG. 3). This was somewhat surprising with respect to previous studies in different cell types in which reconstitution by a protease-dead IRAP variant fully restored vesicular distribution and endosomal trafficking in IRAPko cells, suggesting that the enzymatic activity was dispensable for IRAP-mediated trafficking functions^{43,45}. We therefore hypothesized that in addition to blocking its catalytic activity, HI-419 may also affect the stability of IRAP. In agreement with this hypothesis, using intracellular flow cytometry staining, we detected decreased expression levels of IRAP and VAMP3 starting from 24 h of HFI-419 treatment (data not shown), suggesting that ligation of the inhibitor HFI-419 induced IRAP degradation, ultimately hampering TNF-α secretion.

### Discussion

In the present study we demonstrate that IRAP controls late-phase pro-inflammatory cytokine secretion in mast cells in vitro and in vivo. We find that in Ca²⁺-activated IRAPko mast cells, secretion of TNF-α and IL-6 was reduced compared to wt cells. This was due to a trafficking defect rather than reduced cytokine synthesis because intracellular cytokine levels were comparable between wt and IRAPko cells in response to intracellular Ca²⁺ triggers. The observed inhibition of cytokine secretion was in the order of 50%, and this reduction was physiologically relevant, as IRAPko mice showed milder disease phenotypes in two experimental models of TNF-α-dependent pathologies, namely CAIA and cisplatin-induced acute kidney injury.

Previous reports have implicated the recycling endosome-related SNARE VAMP3 in the 25 constitutive secretion pathway in mast cells 17,19 and macrophages²¹. We extend these findings by showing that, in the absence of IRAP, the amount of VAMP3 colocalizing with Stx4, the SNARE involved in vesicle fusion with the plasma membrane, was reduced. This was most likely due to the observed reduction in the formation or stabilization of Stx6+ post-Golgi carriers in IRAPko cells.

Consistently, a Golgi export assay confirmed that, in IRAPko mast cells, de novo synthesized TNF-α persisted in the Golgi for longer periods of time. These results collectively hint to a role for IRAP in formation of Stx6+ carriers in charge of TNF-α and IL-6 transport at the TGN. The prevailing localization of IRAP to a sequestered pool of cytoplasmic vesicles in the steady-state is difficult to reconcile with a role as sorting effector at the TGN. However, IRAP vesicles are mobilized in response to specific activation signals which induce cleavage of the cytosolic retention protein TUG and transport of IRAP to the cell surface³⁴. Importantly, it has been suggested that under prolonged stimulation, IRAP recycles through endosomes and Golgi back to the plasma membrane without transit via the retention pool^{38,54} In the present study, we identify this exocytic/recycling pathway of IRAP as overlapping with constitutive cytokine secretion. Moreover, the defective TNF-α secretion in the presence of endocytosis inhibitors that was specifically observed in IRAPwt cells suggests that IRAP endocytosis is required for efficient post-Golgi trafficking of cytokines. Taken together, we suggest that activation of mast cells results in IRAP mobilization to the plasma membrane, re-internalization and retrieval to the TGN where it functions as a sorting receptor for cytokines and possibly other molecules secreted along the constitutive pathway.

Post-Golgi transport vesicles containing TNF-α and IL-6 are formed through fission of tubular compartments from the TGN. Budding of these tubular carriers occurs from different TGN subdomains and depends on different coiled-coil golgins⁵⁶. For instance, the transporters involved in TNF-α exit from the Golgi are positive for golgin-245/p230⁵⁷, while the sorting and export of Glut4 and IRAP to the sequestered GSV pool in adipocytes is golgin-160 dependent. Importantly, upon depletion of golgin-160, Glut4 is routed to the PM⁵⁸. We therefore speculate that under persistent activation, the interaction between IRAP and golgin-160 is abrogated, possibly through a post-translational modification of IRAP, changing the post-Golgi trafficking of IRAP and sorting it into a distinct, most likely golgin-245-dependent pathway to the PM.

Interestingly, IL-10 secretion was not affected by the loss of IRAP. Studies in macrophages, where the secretion pathways of TNF-α, IL-6 and IL-10 have been studied in detail, revealed that while these three cytokines may use a common route from the TGN to the recycling endosome, IL-10 alternatively uses a distinct post-Golgi pathway that overlaps with trafficking of the lipoprotein ApoE⁵⁹. Based on our results we suggest that, at least in mast cells, the portion of IL-10 trafficking along the same IRAP-dependent pathway as IL-6 and TNF-α is minor.

With respect to regulated exocytosis, we observed increased secretory granule release in mast cells in the absence of IRAP. Consistently, more VAMP8 staining was observed on Stx4-positive membrane domains, indicating increased fusion events between secretory granules and the plasma membrane in IRAPko as compared to wt cells.

This dichotomy of impaired constitutive trafficking and augmented secretory lysosome/granule trafficking is reminiscent of a report on sortilin ko cytotoxic T and NK cells. In these cells, sortilin has been suggested to regulate both VAMP7 targeting to lysosomes and constitutive secretion of IFNγ (but not TNF-α)⁵³.

Although a direct role for IRAP in the lysosomal targeting or degradation of Vamp8 seems unlikely considering the absence of colocalization between those two proteins, we cannot exclude IRAP-dependent trafficking of proteins that negatively regulate VAMP8 degradation. Alternatively, considering that the same SNARE docking and fusion machinery is used for exocytosis of VAMP3+ vesicles and VAMP8+ granules, diminished abundance of VAMP3+ carriers at the PM might leave more Stx4-SNAP23 molecules available for SNARE complex formation with VAMP8, ultimately augmenting the VAMP8-dependent degranulation rate. Moreover, the activity of several VAMP family members including VAMP8 can be regulated via phosphorylation through PKC which terminates the degranulation response 6°. This regulation likely prevents dangerous consequences of excessive degranulation from mast cells, notably anaphylactic shock. In contrast, this level of regulation is lacking for VAMP3 due to the absence of a phosphorylation motif 6°, suggesting that other regulatory mechanisms may exist. The implication of signal-responsive IRAP endosomes in VAMP3-dependent exocytosis might constitute such a mechanism, i.e. linking extracellular cues to cytokine trafficking.

IRAP protein expression was induced by LPS, in agreement with a previous report that showed IRAP mRNA induction by LPS and IFN-γ but not TGF-β in macrophages⁶¹. These findings suggest that IRAP endosomes are part of a transcriptionally regulated trafficking machinery that is induced by pro-inflammatory environmental cues. Particularly in the light of a recently emerging polarization concept for mast cell functions in inflammation and cancer, in analogy to macrophage M1 vs M2 polarization, the transcriptional regulation of IRAP endosomes deserves further exploration.

We also showed that macrophages depend on IRAP expression for TNF secretion. Considering the broad expression profile of IRAP amongst immune cells, IRAP might regulate cytokine secretion in other cell types, especially those that need to maintain a temporal or spatial segregation between regulated secretion of stored granules and constitutive secretion, such as platelets, cytotoxic T cells, NK cells and basophils.

Finally, IRAP expression was reduced using the chemical inhibitor HI-419. Considering that HI-419 binds to the substrate binding pocket in the intraluminal region of IRAP⁶², the diminution in protein levels strongly suggest conformational effects in trans acting on the cytosolic tail of IRAP 63, which contains specific motifs for the regulated trafficking and interaction of IRAP with several proteins involved in vesicular trafficking such as formins^{44,64}, tankyrase⁶⁵ and p115⁶⁶. We have previously shown that the loss of IRAP anchoring to the actin cytoskeleton promoted destabilization and degradation of IRAP endosomes through rapid retrograde dynein-mediated transport and fusion with lysosomes^{42,44} In summary, our results identify IRAP as a transcriptionally regulated hub of late phase-cytokine secretion in mast cells and a potential target for anti-inflammatory drug development.

### REFERENCES

Throughout this application, various references describe the state of the art to which this invention pertains. The disclosures of these references are hereby incorporated by reference into the present disclosure.
1. Kandere-Grzybowska, K. et al. IL-1 induces vesicular secretion of IL-6 without degranulation from human mast cells. J. Immunol. Baltim. Md 1950 171, 4830-4836 (2003).
2. Qiao, H., Andrade, M. V., Lisboa, F. A., Morgan, K. & Beaven, M. A. FcepsilonR1 and toll-like receptors mediate synergistic signals to markedly augment production of inflammatory cytokines in murine mast cells. Blood 107, 610-618 (2006).
3. Moon, T. C., Befus, A. D. & Kulka, M. Mast cell mediators: their differential release and the secretory pathways involved. Front. Immunol. 5, 569 (2014).
4. Wernersson, S. & Pejler, G. Mast cell secretory granules: armed for battle. Nat. Rev. Immunol. 14, 478-494 (2014)
5. Mukai, K., Tsai, M., Saito, H. & Galli, S. J. Mast cells as sources of cytokines, chemokines, and growth factors. Immunol. Rev. 282, 121-150 (2018).
6. Gordon, J. R. & Galli, S. J. Mast cells as a source of both preformed and immunologically inducible TNF-alpha/cachectin. Nature 346, 274-276 (1990).
7. De Filippo, K. et al. Mast cell and macrophage chemokines CXCL1/CXCL2 control the early stage of neutrophil recruitment during tissue inflammation. Blood 121, 4930-4937 (2013).
8. Dudeck, J. et al. Directional mast cell degranulation of tumor necrosis factor into blood vessels primes neutrophil extravasation. Immunity 54, 468-483.e5 (2021).
9. de Vries, V. C. et al. Mast cells condition dendritic cells to mediate allograft tolerance. Immunity 35, 550-561 (2011).
10. Dudeck, J. et al. Mast-Cell-Derived TNF Amplifies CD8(+) Dendritic Cell Functionality and CD8(+) T Cell Priming. Cell Rep. 13, 399-411 (2015).
11. Rijnierse, A., Koster, A. S., Nijkamp, F. P. & Kraneveld, A. D. TNF-alpha is crucial for the development of mast cell-dependent colitis in mice. Am. J. Physiol. Gastrointest. Liver Physiol. 291, G969-976 (2006).
12. Summers, S. A. et al. Mast cells mediate acute kidney injury through the production of TNF. J. Am. Soc. Nephrol. JASN 22, 2226-2236 (2011).
13. Lee, D. M. et al. Mast cells: a cellular link between autoantibodies and inflammatory arthritis. Science 297, 1689-1692 (2002).
14. Nakae, S. et al. Mast cell-derived TNF contributes to airway hyperreactivity, inflammation, and TH2 cytokine production in an asthma model in mice. J. Allergy Clin. Immunol. 120, 48-55 (2007).
15. Shubin, N. J. et al. Proteome analysis of mast cell releasates reveals a role for chymase in the regulation of coagulation factor XIIIA levels via proteolytic degradation. J. Allergy Clin. Immunol. 139, 323-334 (2017).
16. Plum, T. et al. Human Mast Cell Proteome Reveals Unique Lineage, Putative Functions, and Structural Basis for Cell Ablation. Immunity 52, 404-416.e5 (2020).
17. Puri, N. & Roche, P. A. Mast cells possess distinct secretory granule subsets whose exocytosis is regulated by different SNARE isoforms. Proc. Natl. Acad. Sci. U.S.A 105, 2580-2585 (2008).
18. Sander, L. E. et al. Vesicle associated membrane protein (VAMP)-7 and VAMP-8, but not VAMP-2 or VAMP-3, are required for activation-induced degranulation of mature human mast cells. Eur. J. Immunol. 38, 855-863 (2008).
19. Tiwari, N. et al. VAMP-8 segregates mast cell-preformed mediator exocytosis from cytokine trafficking pathways. Blood 111, 3665-3674 (2008).
20. Frank, S. P. C., Thon, K.-P., Bischoff, S. C. & Lorentz, A. SNAP-23 and syntaxin-3 are required for chemokine release by mature human mast cells. Mol. Immunol. 49, 353-358 (2011).
21. Murray, R. Z., Kay, J. G., Sangermani, D. G. & Stow, J. L. A role for the phagosome in cytokine secretion. Science 310, 1492-1495 (2005).
22. Manderson, A. P., Kay, J. G., Hammond, L. A., Brown, D. L. & Stow, J. L. Subcompartments of the macrophage recycling endosome direct the differential secretion of IL-6 and TNFalpha. J. Cell Biol. 178, 57-69 (2007).
23. Pagan, J. K. et al. The t-SNARE syntaxin 4 is regulated during macrophage activation to function in membrane traffic and cytokine secretion. Curr. Biol. CB 13, 156-160 (2003).
24. De Matteis, M. A. & Luini, A. Exiting the Golgi complex. Nat. Rev. Mol. Cell Biol. 9, 273-284 (2008).
25. Kim, T., Gondré-Lewis, M. C., Arnaoutova, I. & Loh, Y. P. Dense-Core Secretory Granule Biogenesis. Physiology 21, 124-133 (2006).
26. Gilfillan, A. M. & Tkaczyk, C. Integrated signalling pathways for mast-cell activation. Nat. Rev. Immunol. 6, 218-230 (2006).
27. Suzuki, R. et al. Molecular editing of cellular responses by the high-affinity receptor for IgE. Science 343, 1021-1025 (2014).
28. Guo, Z., Turner, C. & Castle, D. Relocation of the t-SNARE SNAP-23 from lamellipodia-like cell surface projections regulates compound exocytosis in mast cells. Cell 94, 537-548 (1998).
29. Paumet, F. et al. Soluble NSF attachment protein receptors (SNAREs) in RBL-2H3 mast cells: functional role of syntaxin 4 in exocytosis and identification of a vesicle-associated membrane protein 8-containing secretory compartment. J. Immunol. Baltim. Md 1950 164, 5850-5857 (2000).
30. Olszewski, M. B., Groot, A. J., Dastych, J. & Knol, E. F. TNF trafficking to human mast cell granules: mature chain-dependent endocytosis. J. Immunol. Baltim. Md 1950 178, 5701-5709 (2007).
31. Liao, H., Keller, S. R. & Castle, J. D. Insulin-regulated aminopeptidase marks an antigen-stimulated recycling compartment in mast cells. Traffic Cph. Den. 7, 155-167 (2006).
32. Keller, S. R. The insulin-regulated aminopeptidase: a companion and regulator of GLUT4. Front. Biosci. J. Virtual Libr. 8, s410-420 (2003).
33. Bogan, J. S. & Kandror, K. V. Biogenesis and regulation of insulin-responsive vesicles containing GLUT4. Curr. Opin. Cell Biol. 22, 506-512 (2010).
34. Rubin, B. R. & Bogan, J. S. Intracellular retention and insulin-stimulated mobilization of GLUT4 glucose transporters. Vitam. Horm. 80, 155-192 (2009).
35. Belman, J. P. et al. Acetylation of TUG protein promotes the accumulation of GLUT4 glucose transporters in an insulin-responsive intracellular compartment. J. Biol. Chem. 290, 4447-4463 (2015).
36. Pan, X., Zaarur, N., Singh, M., Morin, P. & Kandror, K. V. Sortilin and retromer mediate retrograde transport of Glut4 in 3T3-L1 adipocytes. Mol. Biol. Cell 28, 1667-1675 (2017).
37. Kandror, K. V. & Pilch, P. F. The sugar is sIRVed: sorting Glut4 and its fellow travelers. Traffic Cph. Den. 12, 665-671 (2011).
38. Bogan, J. S. Regulation of Glucose Transporter Translocation in Health and Diabetes. Annu. Rev. Biochem. 81, 507-532 (2012).
39. Descamps, D. et al. The Role of Insulin Regulated Aminopeptidase in Endocytic Trafficking and Receptor Signaling in Immune Cells. Front. Mol. Biosci. 7, 583556 (2020).
40. Saveanu, L. et al. IRAP identifies an endosomal compartment required for MHC class I cross-presentation. Science 325, 213-217 (2009).
41. Weimershaus, M. et al. Conventional dendritic cells require IRAP-Rab14 endosomes for efficient cross-presentation. J. Immunol. Baltim. Md 1950 188, 1840-1846 (2012).
42. Weimershaus, M. et al. Innate Immune Signals Induce Anterograde Endosome Transport Promoting MHC Class I Cross-Presentation. Cell Rep. 24, 3568-3581 (2018).
43. Weimershaus, M. et al. IRAP Endosomes Control Phagosomal Maturation in Dendritic Cells. Front. Cell Dev. Biol. 8, 585713 (2020).
44. Babdor, J. et al. IRAP+ endosomes restrict TLR9 activation and signaling. Nat. Immunol. 18, 509-518 (2017).
45. Evnouchidou, I. et al. IRAP-dependent endosomal T cell receptor signalling is essential for T cell responses. Nat. Commun. 11, 2779 (2020).
46. McNeil, B. D. et al. Identification of a mast-cell-specific receptor crucial for pseudo-allergic drug reactions. Nature 519, 237-241 (2015).
47. Morimoto, K. et al. Prostaglandin E2-EP3 signaling induces inflammatory swelling by mast cell activation. J. Immunol. Baltim. Md 1950 192, 1130-1137 (2014).
48. Newton, R. C. et al. Biology of TACE inhibition. Ann. Rheum. Dis. 60 Suppl 3, iii25-32 (2001).
49. Kagari, T., Doi, H. & Shimozato, T. The importance of IL-1 beta and TNF-alpha, and the noninvolvement of IL-6, in the development of monoclonal antibody-induced arthritis. J. Immunol. Baltim. Md 1950 169, 1459-1466 (2002).
50. Pitman, N., Asquith, D. L., Murphy, G., Liew, F. Y. & McInnes, I. B. Collagen-induced arthritis is not impaired in mast cell-deficient mice. Ann. Rheum. Dis. 70, 1170-1171 (2011).
51. Cunin, P. et al. Megakaryocytes compensate for Kit insufficiency in murine arthritis. J. Clin. Invest. 127, 1714-1724 (2017).
52. Cenedeze, M. A. et al. The role of toll-like receptor 4 in cisplatin-induced renal injury. Transplant. Proc. 39, 409-411 (2007).
53. Herda, S. et al. The Sorting Receptor Sortilin Exhibits a Dual Function in Exocytic Trafficking of Interferon-7 and Granzyme A in T Cells. Immunity 37, 854-866 (2012).
54. Xu, Y. et al. Dual-mode of insulin action controls GLUT4 vesicle exocytosis. J. Cell Biol. 193, 643-653 (2011).
55. Papakyriakou, A. et al. 3,4-diaminobenzoic acid derivatives as inhibitors of the oxytocinase subfamily of M1 aminopeptidases with immune-regulating properties. J. Med. Chem. 58, 1524-1543 (2015).
56. Gleeson, P. A., Lock, J. G., Luke, M. R. & Stow, J. L. Domains of the TGN: coats, tethers and G proteins. Traffic Cph. Den. 5, 315-326 (2004).
57. Lieu, Z. Z. et al. A trans-Golgi network golgin is required for the regulated secretion of TNF in activated macrophages in vivo. Proc. Natl. Acad. Sci. U.S.A 105, 3351-3356 (2008).
58. Williams, D., Hicks, S. W., Machamer, C. E. & Pessin, J. E. Golgin-160 is required for the Golgi membrane sorting of the insulin-responsive glucose transporter GLUT4 in adipocytes. Mol. Biol. Cell 17, 5346-5355 (2006).
59. Stanley, A. C. et al. Recycling endosome-dependent and -independent mechanisms for IL-10 secretion in LPS-activated macrophages. J. Leukoc. Biol. 92, 1227-1239 (2012).
60. Malmersjö, S. et al. Phosphorylation of residues inside the SNARE complex suppresses secretory vesicle fusion. EMBO J. 35, 1810-1821 (2016).
61. Nikolaou, A. et al. Presence and regulation of insulin-regulated aminopeptidase in mouse macrophages. J. Renin-Angiotensin-Aldosterone Syst. JRAAS 15, 466-479 (2014).
62. Albiston, A. L. et al. Identification and characterization of a new cognitive enhancer based on inhibition of insulin-regulated aminopeptidase. FASEB J. Off. Publ. Fed. Am. Soc. Exp. Biol. 22, 4209-4217 (2008).
63. Ascher, D. B. et al. Regulation of insulin-regulated membrane aminopeptidase activity by its C-terminal domain. Biochemistry 50, 2611-2622 (2011).
64. Tojo, H. et al. The Formin family protein, formin homolog overexpressed in spleen, interacts with the insulin-responsive aminopeptidase and profilin IIa. Mol. Endocrinol. Baltim. Md 17, 1216-1229 (2003).
65. Chi, N. W. & Lodish, H. F. Tankyrase is a golgi-associated mitogen-activated protein kinase substrate that interacts with IRAP in GLUT4 vesicles. J. Biol. Chem. 275, 38437-38444 (2000).
66. Hosaka, T. et al. p115 Interacts with the GLUT4 vesicle protein, IRAP, and plays a critical role in insulin-stimulated GLUT4 translocation. Mol. Biol. Cell 16, 2882-2890 (2005).

1. A method of treating an inflammatory disease in a patient in need thereof comprising administering to the patient a therapeutically effective amount of an IRAP inhibitor.

2. The method of claim 1 wherein the inflammatory disease is selected from the group arthritis, rheumatoid arthritis, acute arthritis, chronic rheumatoid arthritis, gouty arthritis, acute gouty arthritis, chronic inflammatory arthritis, degenerative arthritis, infectious arthritis, Lyme arthritis, proliferative arthritis, psoriatic arthritis, vertebral arthritis, and juvenile-onset rheumatoid arthritis, osteoarthritis, arthritis chronica progrediente, arthritis deformans, polyarthritis chronica primaria, reactive arthritis, and ankylosing spondylitis), inflammatory hyperproliferative skin diseases, psoriasis such as plaque psoriasis, gutatte psoriasis, pustular psoriasis, and psoriasis of the nails, dermatitis including contact dermatitis, chronic contact dermatitis, allergic dermatitis, allergic contact dermatitis, dermatitis herpetiformis, and atopic dermatitis, x-linked hyper IgM syndrome, urticaria such as chronic allergic urticaria and chronic idiopathic urticaria, including chronic autoimmune urticaria, polymyositis/dermatomyositis, juvenile dermatomyositis, toxic epidermal necrolysis, scleroderma, systemic scleroderma, sclerosis, systemic sclerosis, multiple sclerosis (MS), spino-optical MS, primary progressive MS (PPMS), relapsing remitting MS (RRMS), progressive systemic sclerosis, atherosclerosis, arteriosclerosis, sclerosis disseminata, and ataxic sclerosis, inflammatory bowel disease (IBD), Crohn's disease, colitis, ulcerative colitis, colitis ulcerosa, microscopic colitis, collagenous colitis, colitis polyposa, necrotizing enterocolitis, transmural colitis, autoimmune inflammatory bowel disease, pyoderma gangrenosum, erythema nodosum, primary sclerosing cholangitis, episcleritis, respiratory distress syndrome, adult or acute respiratory distress syndrome (ARDS), meningitis, inflammation of all or part of the uvea, iritis, choroiditis, an autoimmune hematological disorder, rheumatoid spondylitis, sudden hearing loss, IgE-mediated diseases such as anaphylaxis and allergic and atopic rhinitis, encephalitis, Rasmussen's encephalitis, limbic and/or brainstem encephalitis, uveitis, anterior uveitis, acute anterior uveitis, granulomatous uveitis, nongranulomatous uveitis, phacoantigenic uveitis, posterior uveitis, autoimmune uveitis, glomerulonephritis (GN), idiopathic membranous GN or idiopathic membranous nephropathy, membrano- or membranous proliferative GN (MPGN), rapidly progressive GN, allergic conditions, autoimmune myocarditis, leukocyte adhesion deficiency, systemic lupus erythematosus (SLE) or systemic lupus erythematodes such as cutaneous SLE, subacute cutaneous lupus erythematosus, neonatal lupus syndrome (NLE), lupus erythematosus disseminatus, lupus (including nephritis, cerebritis, pediatric, non-renal, extra-renal, discoid, alopecia), juvenile onset (Type I) diabetes mellitus, including pediatric insulin-dependent diabetes mellitus (IDDM), adult onset diabetes mellitus (Type II diabetes), autoimmune diabetes, idiopathic diabetes insipidus, immune responses associated with acute and delayed hypersensitivity mediated by cytokines and T-lymphocytes, tuberculosis, sarcoidosis, granulomatosis, lymphomatoid granulomatosis, Wegener's granulomatosis, agranulocytosis, vasculitides, including vasculitis, large vessel vasculitis, polymyalgia rheumatica, giant cell (Takayasu's) arteritis, medium vessel vasculitis, Kawasaki's disease, polyarteritis nodosa, microscopic polyarteritis, CNS vasculitis, necrotizing, cutaneous, hypersensitivity vasculitis, systemic necrotizing vasculitis, and ANCA-associated vasculitis, such as Churg-Strauss vasculitis or syndrome (CSS), temporal arteritis, aplastic anemia, autoimmune aplastic anemia, Coombs positive anemia, Diamond Blackfan anemia, hemolytic anemia or immune hemolytic anemia including autoimmune hemolytic anemia (AIHA), pernicious anemia (anemia perniciosa), Addison's disease, pure red cell anemia or aplasia (PRCA), Factor VIII deficiency, hemophilia A, autoimmune neutropenia, pancytopenia, leukopenia, diseases involving leukocyte diapedesis, CNS inflammatory disorders, multiple organ injury syndrome such as those secondary to septicemia, trauma or hemorrhage, antigen-antibody complex-mediated diseases, anti-glomerular basement membrane disease, anti-phospholipid antibody syndrome, allergic neuritis, Bechet's or Behcet's disease, Castleman's syndrome, Goodpasture's syndrome, Reynaud's syndrome, Sjogren's syndrome, Stevens-Johnson syndrome, pemphigoid such as pemphigoid bullous and skin pemphigoid, pemphigus, optionally Pemphigus vulgaris, Pemphigus foliaceus, pemphigus mucus-membrane pemphigoid, pemphigus erythematosus, autoimmune polyendocrinopathies, Reiter's disease or syndrome, immune complex nephritis, antibody-mediated nephritis, neuromyelitis optica, polyneuropathies, chronic neuropathy, IgM polyneuropathies, IgM-mediated neuropathy, thrombocytopenia, thrombotic thrombocytopenic purpura (TTP), idiopathic thrombocytopenic purpura (ITP), autoimmune orchitis and oophoritis, primary hypothyroidism, hypoparathyroidism, autoimmune thyroiditis, Hashimoto's disease, chronic thyroiditis (Hashimoto's thyroiditis); subacute thyroiditis, autoimmune thyroid disease, idiopathic hypothyroidism, Grave's disease, polyglandular syndromes such as autoimmune polyglandular syndromes (or polyglandular endocrinopathy syndromes), paraneoplastic syndromes, including neurologic paraneoplastic syndromes such as Lambert-Eaton myasthenic syndrome or Eaton-Lambert syndrome, stiff-man or stiff-person syndrome, encephalomyelitis, allergic encephalomyelitis, experimental allergic encephalomyelitis (EAE), myasthenia gravis, thymoma-associated myasthenia gravis, cerebellar degeneration, neuromyotonia, opsoclonus or opsoclonus myoclonus syndrome (OMS), and sensory neuropathy, multifocal motor neuropathy, Sheehan's syndrome, autoimmune hepatitis, chronic hepatitis, lupoid hepatitis, giant cell hepatitis, chronic active hepatitis or autoimmune chronic active hepatitis, lymphoid interstitial pneumonitis, bronchiolitis obliterans (non-transplant) vs NSIP, Guillain-Barre syndrome, Berger's disease (IgA nephropathy), idiopathic IgA nephropathy, linear IgA dermatosis, primary biliary cirrhosis, pneumonocirrhosis, autoimmune enteropathy syndrome, Celiac disease, Coeliac disease, celiac sprue (gluten enteropathy), refractory sprue, idiopathic sprue, cryoglobulinemia, amylotrophic lateral sclerosis (ALS; Lou Gehrig's disease), coronary artery disease, autoimmune ear disease such as autoimmune inner ear disease (AGED), autoimmune hearing loss, opsoclonus myoclonus syndrome (OMS), polychondritis such as refractory or relapsed polychondritis, pulmonary alveolar proteinosis, amyloidosis, scleritis, a non-cancerous lymphocytosis, a primary lymphocytosis, which includes monoclonal B cell lymphocytosis, optionally benign monoclonal gammopathy or monoclonal garnmopathy of undetermined significance, MGUS, peripheral neuropathy, paraneoplastic syndrome, channelopathies such as epilepsy, migraine, arrhythmia, muscular disorders, deafness, blindness, periodic paralysis, and channelopathies of the CNS, autism, inflammatory myopathy, focal segmental glomerulosclerosis (FSGS), endocrine opthalmopathy, uveoretinitis, chorioretinitis, autoimmune hepatological disorder, fibromyalgia, multiple endocrine failure, Schmidt's syndrome, adrenalitis, gastric atrophy, presenile dementia, demyelinating diseases such as autoimmune demyelinating diseases, diabetic nephropathy, Dressler's syndrome, alopecia greata, CREST syndrome (calcinosis, Raynaud's phenomenon, esophageal dysmotility, sclerodactyl), and telangiectasia), male and female autoimmune infertility, mixed connective tissue disease, Chagas' disease, rheumatic fever, recurrent abortion, farmer's lung, erythema multiforme, post-cardiotomy syndrome, Cushing's syndrome, bird-fancier's lung, allergic granulomatous angiitis, benign lymphocytic angiitis, Alport's syndrome, alveolitis such as allergic alveolitis and fibrosing alveolitis, interstitial lung disease, transfusion reaction, leprosy, malaria, leishmaniasis, kypanosomiasis, schistosomiasis, ascariasis, aspergillosis, Sampter's syndrome, Caplan's syndrome, dengue, endocarditis, endophthalmitis, erythema elevatum et diutinum, erythroblastosis fetalis, eosinophilic faciitis, Shulman's syndrome, Felty's syndrome, flariasis, cyclitis such as chronic cyclitis, heterochronic cyclitis, iridocyclitis, or Fuch's cyclitis, Henoch-Schonlein purpura, human immunodeficiency virus (HIV) infection, echovirus infection, cardiomyopathy, Alzheimer's disease, parvovirus infection, rubella virus infection, post-vaccination syndromes, congenital rubella infection, Epstein-Barr virus infection, mumps, Evan's syndrome, autoimmune gonadal failure, Sydenham's chorea, post-streptococcal nephritis, thromboangitis ubiterans, thyrotoxicosis, tabes dorsalis, chorioiditis, giant cell polymyalgia, endocrine ophthamopathy, chronic hypersensitivity pneumonitis, keratoconjunctivitis sicca, epidemic keratoconjunctivitis, idiopathic nephritic syndrome, minimal change nephropathy, benign familial and ischemia-reperfusion injury, retinal autoimmunity, joint inflammation, bronchitis, chronic obstructive airway disease, silicosis, aphthae, aphthous stomatitis, arteriosclerotic disorders, aspermiogenese, autoimmune hemolysis, Boeck's disease, cryoglobulinemia, Dupuytren's contracture, endophthalmia phacoanaphylactica, enteritis allergica, erythema nodosum leprosum, idiopathic facial paralysis, chronic fatigue syndrome, febris rheumatica, Hamman-Rich's disease, sensoneural hearing loss, haemoglobinuria paroxysmatica, hypogonadism, ileitis regionalis, leucopenia, mononucleosis infectiosa, traverse myelitis, primary idiopathic myxedema, nephrosis, ophthalmia symphatica, orchitis granulomatosa, pancreatitis, polyradiculitis acuta, pyoderma gangrenosum, Quervain's thyreoiditis, acquired splenic atrophy, infertility due to antispermatozoan antobodies, non-malignant thymoma, vitiligo, SCID and Epstein-Barr virus-associated diseases, acquired immune deficiency syndrome (AIDS), parasitic diseases such as Lesihmania, toxic-shock syndrome, food poisoning, conditions involving infiltration of T cells, leukocyte-adhesion deficiency, immune responses associated with acute and delayed hypersensitivity mediated by cytokines and T-lymphocytes, diseases involving leukocyte diapedesis, multiple organ injury syndrome, antigen-antibody complex-mediated diseases, antiglomerular basement membrane disease, allergic neuritis, autoimmune polyendocrinopathies, oophoritis, primary myxedema, autoimmune atrophic gastritis, sympathetic ophthalmia, rheumatic diseases, mixed connective tissue disease, nephrotic syndrome, insulitis, polyendocrine failure, peripheral neuropathy, autoimmune polyglandular syndrome type I, adult-onset idiopathic hypoparathyroidism (AOIH), alopecia totalis, dilated cardiomyopathy, epidermolisis bullosa acquisita (EBA), hemochromatosis, myocarditis, nephrotic syndrome, primary sclerosing cholangitis, purulent or nonpurulent sinusitis, acute or chronic sinusitis, ethmoid, frontal, maxillary, or sphenoid sinusitis, an eosinophil-related disorder such as eosinophilia, pulmonary infiltration eosinophilia, eosinophilia-myalgia syndrome, Loffler's syndrome, chronic eosinophilic pneumonia, tropical pulmonary eosinophilia, bronchopneumonic aspergillosis, aspergilloma, or granulomas containing eosinophils, anaphylaxis, seronegative spondyloarthritides, polyendocrine autoimmune disease, sclerosing cholangitis, sclera, episclera, chronic mucocutaneous candidiasis, Bruton's syndrome, transient hypogammaglobulinemia of infancy, Wiskott-Aldrich syndrome, ataxia telangiectasia, autoimmune disorders associated with collagen disease, rheumatism, neurological disease, ischemic re-perfusion disorder, reduction in blood pressure response, vascular dysfunction, antgiectasis, tissue injury, cardiovascular ischemia, hyperalgesia, cerebral ischemia, and disease accompanying vascularization, allergic hypersensitivity disorders, glomerulonephritides, reperfusion injury, reperfusion injury of myocardial or other tissues, dermatoses with acute inflammatory components, acute purulent meningitis or other central nervous system inflammatory disorders, ocular and orbital inflammatory disorders, granulocyte transfusion-associated syndromes, cytokine-induced toxicity, acute serious inflammation, chronic intractable inflammation, pyelitis, pneumonocirrhosis, diabetic retinopathy, diabetic large-artery disorder, endarterial hyperplasia, peptic ulcer, valvulitis, and endometriosis.

3. The method of claim 1 wherein the inflammatory disease is an allergic disorder, asthma, anaphylaxis, or an inflammatory diseases that is secondary to a treatment with an immune checkpoint inhibitor.

4. The method of claim 1 wherein the inflammatory disease is chemotherapy induced inflammation.

5. The method of claim 1 wherein the IRAP inhibitor has a structure according to Formula (I): 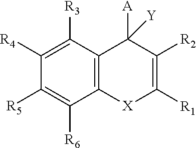
A is aryl, heteroaryl carbocyclyl or heterocyclyl, each of which may be optionally substituted, when R1 is NHCOR₈; or quinolinyl, isoquinolinyl, cinnolinyl, quinazolinyl, quinoxalinyl, 1,8-naphthyridyl, phthalazinyl or pteridinyl, each of which may be optionally substituted, when R1 is NR₇R₈, NHCOR₈, N(COR₈)₂, N(COR₇)(COR₈), N═CHOR₈ or N═CHR₈;
X is 0, NR′ or S, wherein R′ is hydrogen, optionally substituted alkyl, optionally substituted alkenyl, optionally substituted alkynyl, optionally substituted aryl, optionally substituted acyl, optionally substituted heteroaryl, optionally substituted carbocyclyl or optionally substituted heterocyclyl;
R₇ and R₈ are independently selected from hydrogen, optionally substituted alkyl, optionally substituted aryl, or R₇ and R₈, together with the nitrogen atom to which they are attached form a 3-8-membered ring which may be optionally substituted;
R2 is CN, CO₂R₉, C(O)O(O)R₉, C(O)R₉ or C(O)NR₉R₁₀ wherein R₉ and R₁₀ are independently selected from alkyl, alkenyl, alkynyl, aryl, heteroaryl, carbocyclyl, heterocyclyl, each of which may be optionally substituted, and hydrogen; or R₉ and R₁₀ together with the nitrogen atom to which they are attached, form a 3-8-membered ring which may be optionally substituted;
R₃-R₆ are independently selected from hydrogen, halo, nitro, cyano alkyl, alkenyl, alkynyl, aryl, heteroaryl, heterocyclyl, carbocyclyl, hydroxy, alkoxy, alkenyloxy, alkynyloxy, alkynyloxy, aryloxy, heteroaryloxy, heterocyclyloxy, amino, acyl, acyloxy, carboxy, carboxyester, methylenedioxy, amido, thio, alkylthio, alkenylthio, alkynylthio, arylthio, heteroarylthio, heterocyclylthio, carbocyclylthio, acylthio and azido, each of which may be optionally substituted where appropriate, or any two adjacent R₃-R₆, together with the atoms to which they are attached, form a 3-8-membered ring which may be optionally substituted; and
Y is hydrogen or C₁₋₁₀ alkyl, or a pharmaceutically acceptable salt or solvate thereof.

6. The method of claim 1 wherein the IRAP inhibitor has the structure:

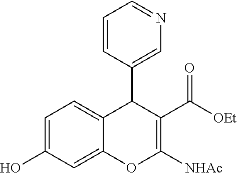 [SMILES: CCOC(=O)C1=C(NC(C)=O)Oc2cc(O)ccc2C1c1cccnc1]

7. The method of claim 1 wherein the IRAP inhibitor has a structure according to Formula (II): 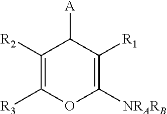
wherein:
A is selected from alkyl, alkenyl, alkynyl, aryl, arylalkyl, heteroaryl, heteroarylalkyl, carbocyclyl, carbocyclylalkyl, each of which may be optionally substituted;
RA and RB are independently selected from hydrogen, alkyl and acyl;
R1 is selected from CN or CO2RC;
R2 is selected from CO2RC and acyl;
R3 is selected from alkyl, alkenyl, alkynyl, aryl, arylalkyl, heteroaryl, heteroarylalkyl, carbocyclyl, carbocyclylalkyl, each of which may be optionally substituted; or
R2 and R3 together form a 5-6-membered saturated keto-carbocyclic ring: 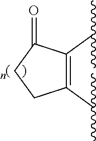
wherein n is 1 or 2; and which ring may be optionally substituted one or more times by C1-6 alkyl; or
R2 and R3 together form a 5-membered lactone ring (a) or a 6-membered lactone ring (b) 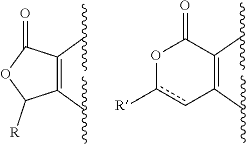
wherein [symbol] is an optional double bond and R′ is alkyl.
Rc is selected from alkyl, alkenyl, alkynyl, aryl, arylalkyl, heteroaryl, heteroarylalkyl, carbocyclyl, carbocyclylalkyl, each of which may be optionally substituted;
or a pharmaceutically acceptable salt, solvate or prodrug thereof.

8. The method of claim 1 wherein the IRA inhibitor has the structure:

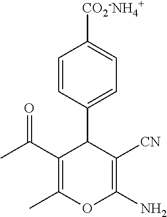

9. The method of claim 1 wherein the IRAP inhibitor has a structure selected from the group consisting of: 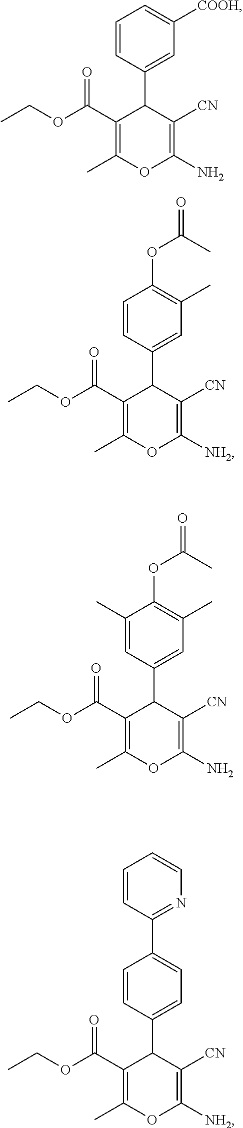 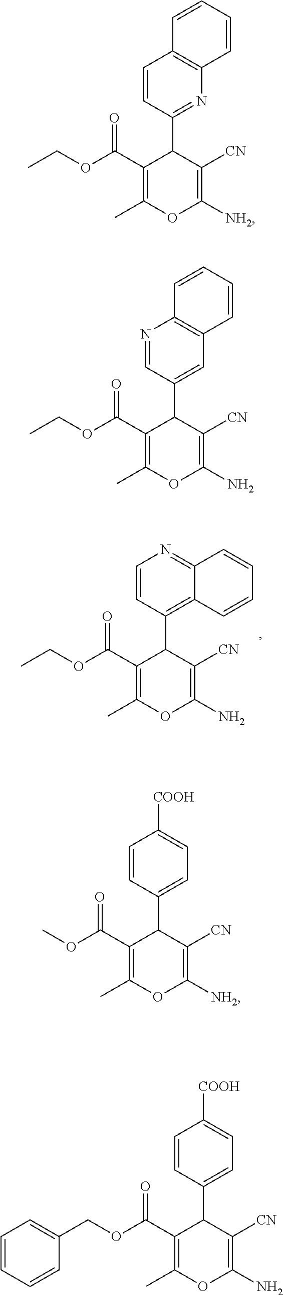 [SMILES: CCOC(=O)C1=C(C)OC(N)=C(C#N)C1c1ccc2ccccc2n1 ; CCOC(=O)C1=C(C)OC(N)=C(C#N)C1c1cnc2ccccc2c1 ; CCOC(=O)C1=C(C)OC(N)=C(C#N)C1c1ccnc2ccccc12 ; COC(=O)C1=C(C)OC(N)=C(C#N)C1c1ccc(C(=O)O)cc1 ; CC1=C(C(=O)OCc2ccccc2)C(c2ccc(C(=O)O)cc2)C(C#N)=C(N)O1] 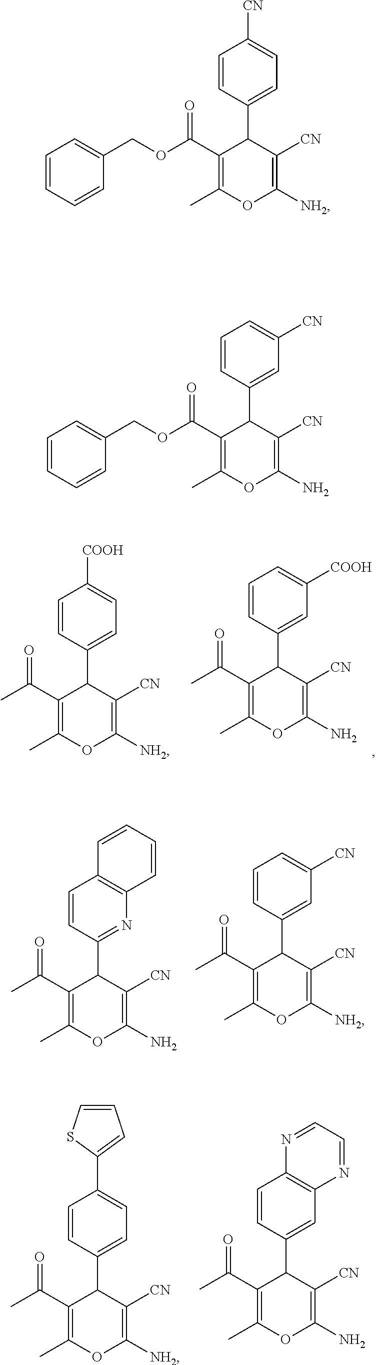 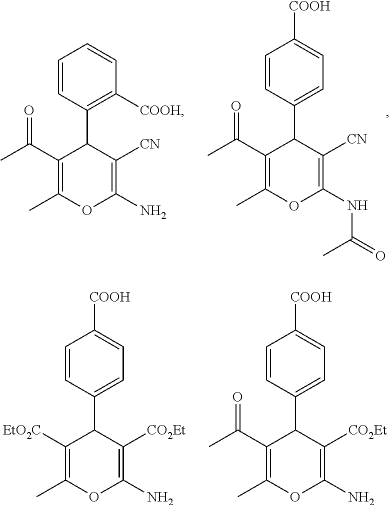 [SMILES: CC(=O)C1=C(C)OC(N)=C(C#N)C1c1ccccc1C(=O)O ; CC(=O)NC1=C(C#N)C(c2ccc(C(=O)O)cc2)C(C(C)=O)=C(C)O1 ; CCOC(=O)C1=C(C)OC(N)=C(C(=O)OCC)C1c1ccc(C(=O)O)cc1 ; CCOC(=O)C1=C(N)OC(C)=C(C(C)=O)C1c1ccc(C(=O)O)cc1]
and/or a pharmaceutically acceptable salt, solvate or prodrug thereof.

10. The method of claim 1 wherein the IRAP inhibitor has a structure according to Formula (III): 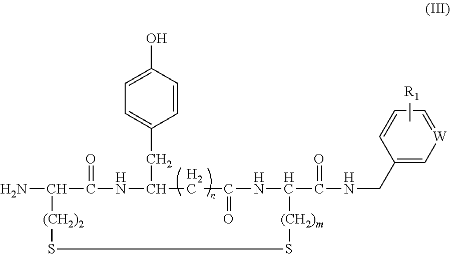
Wherein
R1 is H or CH₂COOH; and
n is 0 or 1; and
m is 1 or 2; and
W is CH or N;
or a pharmaceutically acceptable salt, solvate or prodrug thereof.

11. The method of claim 1 wherein the IRA inhibitor has the structure:

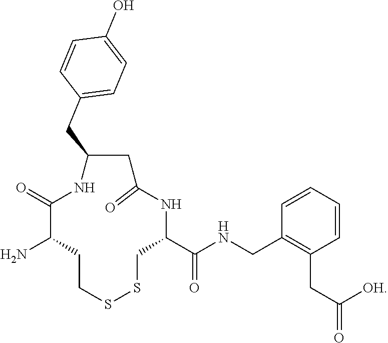 [SMILES: N[C@H]1CCSSC[C@@H](C(=O)NCc2ccccc2CC(=O)O)NC(=O)C[C@H](Cc2ccc(O)cc2)NC1=O]

12. The method of claim 1 wherein the IRAP inhibitor has the structure

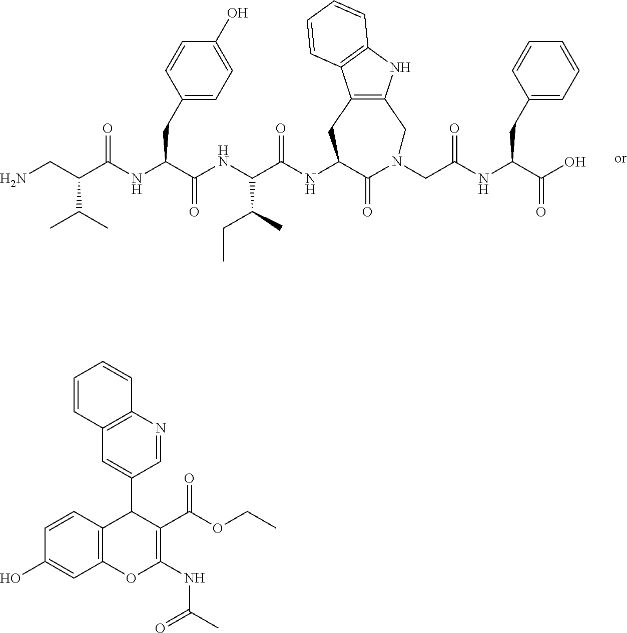

13. The method of claim 1 wherein the MAP inhibitor is (±)-Ethyl-2-acetamido-7-hydroxy-4-(pyridin-3-yl)-4H-chromene-3-carboxylate (HFI-419)

14. The method of claim 1 wherein the IRAP inhibitor is an inhibitor of IRAP expression such as a siRNA or antisense oligonucleotide.

15. The method of claim 1 wherein the IRAP inhibitor leads to the destabilization and/or degradation of IRAP.


SEQUENCE LISTING (1 sequence)

SEQ ID NO: 1 - protein, 1025 aa, Homo sapiens
MEPFTNDRLQLPRNMIENSMFEEEPDVVDLAKEPCLHPLEPDEVEYEPRGSRLLVRGLGEHEMEEDEEDYESSAKLLGMSFMNRSSGLRNSATGYRQSPDGACSVPSARTMVVCAFVIVVAVSVIMVIYLLPRCTFTKEGCHKKNQSIGLIQPFATNGKLFPWAQIRLPTAVVPLRYELSLHPNLTSMTFRGSVTISVQALQVTWNIILHSTGHNISRVTFMSAVSSQEKQAEILEYAYHGQIAIVAPEALLAGHNYTLKIEYSANISSSYYGFYGFSYTDESNEKKYFAATQFEPLAARSAFPCFDEPAFKATFIIKIIRDEQYTALSNMPKKSSVVLDDGLVQDEFSESVKMSTYLVAFIVGEMKNLSQDVNGTLVSIYAVPEKIGQVHYALETTVKLLEFFQNYFEIQYPLKKLDLVAIPDFEAGAMENWGLLTFREETLLYDSNTSSMADRKLVTKIIAHELAHQWFGNLVTMKWWNDLWLNEGFATFMEYFSLEKIFKELSSYEDFLDARFKTMKKDSLNSSHPISSSVQSSEQIEEMFDSLSYFKGSSLLLMLKTYLSEDVFQHAVVLYLHNHSYASIQSDDLWDSFNEVTNQTLDVKRMMKTWTLQKGFPLVTVQKKGKELFIQQERFFLNMKPEIQPSDTSYLWHIPLSYVTEGRNYSKYQSVSLLDKKSGVINLTEEVLWVKVNINMNGYYIVHYADDDWEALIHQLKINPYVLSDKDRANLINNIFELAGLGKVPLKRAFDLINYLGNENHTAPITEALFQTDLIYNLLEKLGYMDLASRLVTRVFKLLQNQIQQQTWTDEGTPSMRELRSALLEFACTHNLGNCSTTAMKLFDDWMASNGTQSLPTDVMTTVFKVGAKTDKGWSFLLGKYISIGSEAEKNKILEALASSEDVRKLYWLMKSSLNGDNFRTQKLSFIIRTVGRHFPGHLLAWDFVKENWNKLVQKFPLGSYTIQNIVAGSTYLFSTKTHLSEVQAFFENQSEATFRLRCVQEALEVIQLNIQWMEKNLKSLTWWL